(19) United States
(12) Patent Application Publication
(10) Pub. No.: US 2013/0004504 A1
(43) Pub. Date: Jan. 3, 2013

(54) METHOD OF MODULATING PHENYLALANINE HYDROXYLASE STRUCTURE AND FUNCTION

(75) Inventor: Eileen K. JAFFE, Jenkintown, PA (US)

(73) Assignee: Fox Chase Cancer Center, Philadelphia, PA (US)

(21) Appl. No.: 13/537,677

(22) Filed: Jun. 29, 2012

Related U.S. Application Data
(60) Provisional application No. 61/503,823, filed on Jul. 1, 2011.

(51) Int. Cl. C12N 9/96; C07K 16/40; A61K 39/395; G01N 33/573; C40B 30/04

(52) U.S. Cl. 424/146.1; 435/188; 435/7.4; 506/9; 530/389.8

(57) ABSTRACT

A method of affecting a multimeric protein comprising an equilibrium of assembly states, each assembly having a plurality of units, wherein each of the units comprises a first complementary surface and a second complementary surface and wherein the first complementary surface of one unit is associated with the second complementary surface of another unit, provided that the assembly is at least one of different quaternary isoforms under four conditions, wherein the method comprising: applying to the multimeric protein a composition comprising a compound adapted to affect formation of an active form of the multimeric protein; associating the composition with an active form of the multimeric protein; and promoting the multimeric protein to assemble into the active form, thereby affecting the multimeric protein to form the active form, wherein the multimeric protein is phenylalanine hydroxylase.

### BACKGROUND OF THE INVENTION

1. Field of Invention

This invention relates to a method of modulating phenylalanine hydroxylase (PAH) structure and activity. This invention also relates to a method of treating hyperphenylalaninemia or phenylketonuria disease.

2. Description of Related Art

PAH catalyzes the non-heme iron-dependent synthesis of tyrosine from the substrates phenylalanine (Phe) and molecular oxygen using the cofactor tetrahydrobiopterin (BH₄). PAH is a homo-oligomer that participates in a quaternary structure equilibrium consisting of dimers, tetramers, and larger aggregates. The resting form of the enzyme appears to include a low activity conformation. Substrate activation, which is known to be associated with a large conformational change, allows the body an amplified immediate response to increased dietary Phe. Diminished activity of PAH is the most common inborn error of metabolism; the most severe cases result in phenylketonuria (PKU). The interconversion between dimers and tetramers is reversible and defects in multimerization have been associated with PKU. PAH is well defined as an allosteric enzyme whose activation is accompanied by major protein conformational changes.

A spectrum of diseases caused by deficiency in PAH activity result in elevated levels of blood Phe. Observed phenotypes arise from accumulation of high blood Phe, which has been proposed to saturate aromatic amino acid receptors in the brain, thus inhibiting tyrosine and tryptophan access and robbing the brain of the precursors to essential compounds such as dopamine and serotonin. Inadequate control of blood Phe levels during brain development (infancy and childhood) results in severe mental retardation. Inadequate control of blood Phe levels throughout life, including adulthood, also manifests as cognitive and behavioral deficits. Dietary therapy for PAH deficiency, which includes limiting protein (and/or Phe) intake and can include synthetic amino acid or BH₄ supplements, has been in place since the mid-20^{th} century. Dietary control can largely, though not completely, ameliorate the permanent brain damage previously associated with PKU. Dietary compliance falls off during adolescence and adulthood due to the unpalatable nature of the prescribed diet. Additional therapies to further minimize damage during brain development and behavioral consequences throughout life are required. Therapies under development or in use include gene replacement therapy, supplementation with the enzyme phenylalanine ammonia lyase, and treatment with large neutral amino acids. Children and adults living with PAH deficiency, for whom control of the disease is problematic, would benefit from a rationally designed small molecule therapeutic that would minimize brain damage and allow a less restricted diet.

PKU and milder forms of PAH deficiency are autosomal recessive inborn errors of metabolism, requiring an aberrant gene from each parent. In the case of PKU there are ˜500 disease associated mutations that have been mapped. Importantly, the majority of these are not obviously directly involved in catalysis and some have been shown to alter the quaternary structure equilibrium. Genotype/phenotype correlations are complex, underlining the fact that the structural basis for control of PAH activity is inadequately understood.

### BRIEF SUMMARY OF THE INVENTION

Accordingly, the invention provides a method of modulating a multimeric phenylalanine hydroxylase, the method comprising: applying a composition comprising a compound adapted to modulate formation of a multimeric phenylalanine hydroxylase to form an active form; associating the composition with the multimeric phenylalanine hydroxylase; promoting the multimeric protein to assemble into the active form, and thereby activating the multimeric phenylalanine hydroxylase.

Also provided is a method of affecting a multimeric protein comprising an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that: (i) one conformation of said units determines a first quaternary isoform but does not allow formation of another quaternary isoform; (ii) a different conformation of said units determines one of a different quaternary isoform, but does not allow formation of the first quaternary isoform; (iii) the different conformations of said units are in an equilibrium; and (iv) the conformation of said different quaternary isoforms influences a function of said multimeric protein, the method comprising: applying to the multimeric protein a composition comprising a compound adapted to affect formation of an active form of the multimeric protein; associating the composition with an active form of the multimeric protein; and promoting the multimeric protein to assemble into the active form, thereby affecting the multimeric protein to form the active form, wherein said multimeric protein is phenylalanine hydroxylase.

In certain embodiments, said each unit is a member selected from the group consisting of a monomer, a dimer, a trimer, a tetramer, a hexamer, and an octamer.

In certain embodiments, said multimeric protein is phenylalanine hydroxylase comprising four phenylalanine hydroxylase monomers.

Also provided is a method of identifying a compound that modulates formation of a multimeric protein by binding at a site other than an active site of the multimeric protein comprising an assembly having a plurality of units, wherein each of said units comprises a first complementary surface and a second complementary surface and wherein the first complementary surface of one unit is associated with the second complementary surface of another unit, provided that the assembly is at least one of different quaternary isoforms, the method comprising: a) providing at least one multimeric protein with the biochemical function; (b) identifying a compound that binds to the protein; and (c) testing for the ability of the compound to affect the biochemical function of the multimeric protein phenylalanine hydroxylase.

In certain embodiments, said compound binds to a site other than an active site and effects the PAH function.

In certain embodiments, said compound binds to a site other than an active site wherein that site is present in the quaternary isoform of the desired function and not present in the quaternary isoform of the undesired function.

In certain embodiments, said biochemical function of said multimeric protein correlates to a human disease or condition.

In certain embodiments, the effect of the compound on the biochemical function is selected from the group consisting of inhibition, activation, enhancement, modulation, binding, and allosteric effect.

Also provided is a method of identifying a compound adapted to modulate a multimeric protein by binding to a binding site of said multimeric protein, wherein the multimeric protein comprises an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that: (i) one conformation of said units determines a first quaternary isoform but does not allow formation of another quaternary isoform; (ii) a different conformation of said units determines one of a different quaternary isoform, but does not allow formation of the first quaternary isoform; (iii) the different conformations of said units are in an equilibrium; and (iv) the conformation of said different quaternary isoforms influences a function of said multimeric protein, the method comprising: providing a test compound; providing the multimeric protein; contacting the multimeric protein with the test compound; and measuring the equilibrium of units of the multimeric protein, wherein the compound adapted to affect the multimeric protein by binding to a binding site of the multimeric protein is identified when it affects the multimeric protein by binding to a binding site of the multimeric protein and thereby affects an equilibrium of units of the multimeric protein phenylalanine hydroxylase.

In certain embodiments, said compound binds to a site other than an active site and effects the PAH function.

In certain embodiments, said compound binds to a site other than an active site wherein that site is present in the quaternary isoform of the desired function and not present in the quaternary isoform of the undesired function.

In certain embodiments, the unit is a member selected from the group consisting of a monomer, a dimer, a trimer, a tetramer, a hexamer, and an octamer. In certain embodiments, the compound is adapted to affect a function of said multimeric protein. In certain embodiments, the compound is bound to a quaternary isoform having a greater activity. In certain embodiments, the compound activates the enzymatic activity of the multimeric protein. In certain embodiments, the compound is an activator which promotes formation of an active form of the multimeric protein.

Also provided is a method of treating a disease or condition by administering a therapeutically effective amount of the compound identified by the method of identifying a compound that is an activator which promotes formation of an active form of the multimeric protein by binding to a binding site of said multimeric protein, wherein the multimeric protein comprises an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that: (i) one conformation of said units determines a first quaternary isoform but does not allow formation of another quaternary isoform; (ii) a different conformation of said units determines one of a different quaternary isoform, but does not allow formation of the first quaternary isoform; (iii) the different conformations of said units are in an equilibrium; and (iv) the conformation of said different quaternary isoforms influences a function of said multimeric protein, the method comprising: providing a test compound; providing the multimeric protein; contacting the multimeric protein with the test compound; and measuring the equilibrium of units of the multimeric protein, wherein the compound adapted to affect the multimeric protein by binding to a binding site of the multimeric protein is identified when it affects the multimeric protein by binding to a binding site of the multimeric protein and thereby affects an equilibrium of units of the multimeric protein phenylalanine hydroxylase.

Also provided is a compound for use in treating a disease or condition by administering a therapeutically effective amount of the compound, wherein the compound is identified by the method of identifying a compound that is an activator which promotes formation of an active form of the multimeric protein by binding to a binding site of said multimeric protein, wherein the multimeric protein comprises an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that: (i) one conformation of said units determines a first quaternary isoform but does not allow formation of another quaternary isoform; (ii) a different conformation of said units determines one of a different quaternary isoform, but does not allow formation of the first quaternary isoform; (iii) the different conformations of said units are in an equilibrium; and (iv) the conformation of said different quaternary isoforms influences a function of said multimeric protein, the method comprising: providing a test compound; providing the multimeric protein; contacting the multimeric protein with the test compound; and measuring the equilibrium of units of the multimeric protein, wherein the compound adapted to affect the multimeric protein by binding to a binding site of the multimeric protein is identified when it affects the multimeric protein by binding to a binding site of the multimeric protein and thereby affects an equilibrium of units of the multimeric protein phenylalanine hydroxylase

In certain embodiments, the disease or condition is deficiency of PAH activity. In certain embodiments, the disease or condition is hyperphenylalaninemia or, in more severe forms, phenylketonuria.

Also provided is an antibody which selectively binds PAH isoforms 4mer* and 2mer*.

Also provided is an antibody which selectively binds PAH isoforms 4mer and 2mer.

Also provided is a method for predicting whether a hyperphenylalaninemia or phenylketonuria disease patient will respond effectively to treatment with an agent, comprising determining the ratio of PAH isoforms 4mer* and 2mer* to PAH isoforms 4mer and 2mer in a sample and comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* in the sample is greater than the ratio of PAH isoforms 4mer and 2mer in a control, this is an indication that the patient will respond effectively to treatment with the agent.

Also provided is a method for predicting whether a hyperphenylalaninemia or phenylketonuria disease patient will respond effectively to treatment with a compound, comprising: (a) obtaining a sample from a patient/individual; (b) contacting said sample with a detectably-labeled first antibody which selectively binds PAH isoforms 4mer* and 2mer*; (c) contacting said sample with a detectably-labeled second antibody which selectively binds PAH isoforms 4mer and 2mer; (d) incubating the components of steps (b) for a period of time and under conditions sufficient to form an immune complex between said first antibody which selectively binds PAH isoforms 4mer* and 2mer*; (e) incubating the components of steps (c) for a period of time and under conditions sufficient to form an immune complex between said second antibody which selectively binds PAH isoforms 4mer and 2mer; (f) optionally separating unbound antibody from said sample; (g) determining the detectably-labeled PAH isoforms 4mer* and 2mer*; (h) determining the detectably-labeled PAH isoforms 4mer and 2mer; (i) determining the ratio of PAH isoforms 4mer* and 2mer* to PAH isoforms 4mer and 2mer in the sample; (j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will respond effectively to treatment with the compound.

Also provided is a method for predicting the sensitivity of a hyperphenylalaninemia or phenylketonuria disease patient to treatment with an compound, comprising determining the ratio of PAH isoforms 4mer* and 2mer* to PAH isoforms 4mer and 2mer in the sample, comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will be sensitive to treatment with the compound.

Also provided is a method for predicting whether a hyperphenylalaninemia or phenylketonuria disease patient will respond effectively to treatment with a compound, comprising: (a) obtaining a sample from a patient/individual; (b) contacting said sample with a detectably-labeled first antibody which selectively binds PAH isoforms 4mer* and 2mer*; (c) contacting said sample with a detectably-labeled second antibody which selectively binds PAH isoforms 4mer and 2mer; (d) incubating the components of steps (b) for a period of time and under conditions sufficient to form an immune complex between said first antibody which selectively binds PAH isoforms 4mer* and 2mer*; (e) incubating the components of steps (c) for a period of time and under conditions sufficient to form an immune complex between said second antibody which selectively binds PAH isoforms 4mer and 2mer; (f) optionally separating unbound antibody from said sample; (g) determining the detectably-labeled PAH isoforms 4mer* and 2mer*; (h) determining the detectably-labeled PAH isoforms 4mer and 2mer; (i) determining the ratio of PAH isoforms 4mer* and 2mer* to PAH isoforms 4mer and 2mer in the sample; (j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will be sensitive to treatment with the compound.

Also provided is a method for the determination of the ratio of PAH isoforms 4mer* and 2mer* to PAH isoforms 4mer and 2mer in a sample, which comprises determining the ratio of PAH isoforms 4mer* and 2mer* to PAH isoforms 4mer and 2mer in the sample, comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control.

Further provided is a method for the determination of the ratio of PAH isoforms 4mer* and 2mer* to PAH isoforms 4mer and 2mer in a sample, which comprises: (a) obtaining a sample from a patient/individual; (b) contacting said sample with a detectably-labeled first antibody which selectively binds PAH isoforms 4mer* and 2mer*; (c) contacting said sample with a detectably-labeled second antibody which selectively binds PAH isoforms 4mer and 2mer; (d) incubating the components of steps (b) for a period of time and under conditions sufficient to form an immune complex between said first antibody which selectively binds PAH isoforms 4mer* and 2mer*; (e) incubating the components of steps (c) for a period of time and under conditions sufficient to form an immune complex between said second antibody which selectively binds PAH isoforms 4mer and 2mer; (f) optionally separating unbound antibody from said sample; (g) determining the detectably-labeled PAH isoforms 4mer* and 2mer*; (h) determining the detectably-labeled PAH isoforms 4mer and 2mer; (i) determining the ratio of PAH isoforms 4mer* and 2mer* to PAH isoforms 4mer and 2mer in the sample; (j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control.

### BRIEF DESCRIPTION OF SEVERAL VIEWS OF THE DRAWINGS

The invention will be described in conjunction with the following drawings in which like reference numerals designate like elements and wherein:

FIG. 1 is a general representation of the morpheein model of allostery, wherein two different conformations of a monomer assemble into two structurally and functionally different multimers. The wedge is an allosteric regulator molecule that only binds to 1 mer* and 3 mer*, thus pulling the equilibrium towards these forms. The seminal characteristic of the morpheein model of allostery is that a multimer must dissociate and undergo a conformational change prior to reassociating to a structurally and functionally distinct multimer.

FIG. 2 is a representation of human PAH. FIG. 2A is the domain structure of human PAH. Hinge regions are denoted by [symbol] . A disordered region (possible active site lid) is the small white box. FIG. 2B is a ribbon diagram of the inventors' human PAH 4mer* model (colored as in FIG. 2A, 1-33 as strands, 34-410 as ribbon, 411-453 as tube) showing one monomer (fixed orientation) assembling into dimer and tetramer (shown half size), and an alternate orientation of 4mer*.

FIG. 3 shows two examples of ACT domains in the PDB. With the exception of PAH crystal structures, ACT domains present in the PDB all form ACT-ACT dimers that form an interface between two subunits (shown above as black and white). On the left is PDB id 2flf—the ACT-ACT dimer of acetolactate synthase. On the right is PDB id 1 psd—the ACT-ACT dimer of phosphoglycerate dehydrogenase with serine bound at the subunit-subunit interface. Not shown are PDB id 2cdq, where there are two different (tandem) ACT-ACT dimers of aspartate kinase, only one of which contains lysine in the subunit-subunit interface. Also not shown is PDB id 2f06, where there are four ACT-ACT dimers present in protein BT0572 from Bacteroides thetaiotaomicron. Each ACT-ACT dimer in this structure contains one molecule of histidine at the subunit-subunit interface.

FIG. 4 represents one preferred model for allosteric regulation of human PAH (colored as FIG. 2). In this model the regulatory domain undergoes a large spatial reorientation around its hinge regions. In the low activity 4mer* and 2mer*, the regulatory domain partially blocks access to and from the active site ( [symbol] ). In the high activity 2mer and 4mer, the active site is solvent accessible. Rotating the regulatory domain in this way allows ACT subdomains from adjacent dimers to interact to form a binding site for an allosteric Phe. The required reorientation between domains is proposed to be sterically forbidden in the tetramers.

FIG. 5 illustrates alternate PAH tetramer models. In FIG. 5A, the model of PAH 4mer* is as presented in FIG. 2. Arrows denote the ACT:ACT interfaces shown in FIG. 3. FIG. 5B is the described preliminary model of 4mer, displayed as in FIG. 5A, and described in more detailed in Detailed Description of the Invention below. The N-terminal 33 amino acids, although shown, have not yet been properly modeled.

FIG. 6 provides a basis for the reversible formation of higher-order inactive multimers (aggregates) of human PAH (shaded as FIG. 2). Dimerization of the ACT subdomains of PAH in the 2mer* or 4mer* conformations could lead to the reversible formation of higher order low activity aggregates such as the illustrated 6mer*. Such multimerization could produce 8mer*, 10mer*, 12mer*, etc.

FIG. 7 represents preliminary results on purified mammalian PAH. FIG. 7A shows SEC separation of purified human PAH isoforms. Full length human PAH, cleaved from an N-terminal MBP fusion construct, runs at a dimer:tetramer ratio of ˜2:1 on a calibrated Superdex-200 column. With 1 mM Phe added to the running buffer, this ratio is shifted to ˜1:1. Buffers contain 125 mM KCl, which is known to stabilize the tetramer. FIG. 7B represents preliminary kinetic studies on purified rat PAH, monitoring tyrosine fluorescence, showing “order of addition” effects at 300 μM Phe and 75 μM BH4. FIG. 7C shows native PAGE on rat PAH, purified by the affinity method of Shiman (95), suggests an equilibrium containing two different tetramers and a dimer. FIG. 7D (Superdex 200, as in FIG. 7A) shows that purified rat PAH is already predominantly tetramer and this is not altered by Phe. Careful line shape analysis of the profiles suggests the co-existence of two different dimers, with the Phe-activated form eluting slightly earlier. FIG. 7E is a full length rat PAH (˜100 mg/run), run on a 1 ml HiTrap Q column in 30 mM Tris-HCl with a KCl gradient (B=400 mM KCl). 1 mM Phe in the elution buffer dramatically alters the elution profile, but does not suggest species other than tetramer (see FIG. 7D). The addition of Phe to a concentration that is reported to give ˜50% activation shows an equilibrium of structures suggesting that the ion-exchange chromatography (IEC) is resolving two different tetramers. FIG. 7F is native PhastGel Western blot showing the effects of ligands on the rat PAH quaternary structure equilibrium (overnight, RT, ˜1 mg/ml). Lane 1—as purified; Lane 2—1 mM Phe; Lane 3—50 μM Phe; Lane 4—75 μM BH4; Lane 5, 50 μPhe+75 μM BH4.

FIG. 8 represents alternate dissociative and non-dissociative allosteric models for allosteric regulation of human PAH (colored as FIGS. 2 & 4). The proposed models include the reorientation of the regulatory domain relative to the catalytic domain. FIG. 8A is a morpheein model including two conformationally distinct tetramers that must dissociate to the dimer for the required conformational change. FIG. 8B shows a morpheein model that includes an equilibrium of dimers, only one of which can associate to the tetramer. Tetramerization is stabilized by binding an allosteric Phe at the interface between two ACT domains. FIG. 8C represents a classic MWC model that discounts the physiologic relevance of the oft-observed PAH dimer. FIG. 8D offers a model that includes a low activity dimer but allows the conformational change to take place in the context of the tetramer.

FIG. 9 shows disproportionation of chains, which establishes a dissociative mechanism for the PBGS 6mer* to 8mer transition (20). FIG. 9A is an experimental schematic. FIG. 9B is a native PAGE analysis of heteromers. FIG. 9C shows the results of the mass spectroscopy analysis of the tryptic digest showing the mole fractions of Phe12 and Leu12 in the N-terminal peptide. During the 6mer* to 8mer transition during dialysis, Phe12 preferentially partitions to 8mer and Leu12 preferentially remains in 6mer*.

### DETAILED DESCRIPTION OF THE INVENTION

The present invention was prompted by the inventors' studies relating to the application of a new allosteric model to the regulation of porphobilinogen synthase (PBGS). The current invention establishes a morpheein model (5) for the allosteric regulation of PAH. The model minimally consists of a high activity tetramer (4mer), a high activity dimer (2mer), a low activity dimer (2mer*), and a low activity tetramer (4mer*) in the equilibrium:

4mer [symbol] 2mer [symbol] 2mer* [symbol] 4mer*.

This equilibrium includes a mandatory tetramer dissociation event, which is necessary for the interconversion between the conformationally distinct less active and more active forms. The morpheein model of PAH regulation is different from other published models, but builds on a growing view of PKU as a possible conformational disease (6-8). This new model will open novel avenues for small molecule modulation of PAH activity that may prove uniquely beneficial to individuals with PAH deficiencies (9) and may allow development of a diagnostic that can predict which patients are likely to respond to new and existing therapies. PKU is in the Core Panel of the Newborn Screening Panel (10).

The inventors' approach follows methods that were developed to investigate human porphobilinogen synthase (PBGS) (11-19), for which the morpheein equilibrium is 8mer [symbol] 2mer [symbol] 2mer* [symbol] 6mer*; and the rare metabolic disease is ALAD porphyria (20).

First, the invention provides the hypothesis that PAH activity is governed by an equilibrium of quaternary isoforms according to the morpheein model of allosteric regulation (5). The inventors' working model for PAH: 4mer [symbol] 2mer [symbol] 2mer* [symbol] 4mer*, is consistent with the consensus that the physiologically significant multimers of PAH are dimers and tetramers. The inventors have developed chromatographic and electrophoretic methods to separate and discriminate between 4mer, 2mer, 2mer*, and 4mer*. The inventors utilize well established methods for monitoring PAH kinetics (21-28) and focus on correlating known PAH activation phenomena (e.g.(29)) with an nmer [symbol] nmer* transition. The invention provides using established biochemical and biophysical protocols known to discriminate between different conformational states of PAH such as limited tryptic proteolysis, tryptophan fluorescence, surface plasmon resonance, and CD-monitored thermal denaturation (e.g. (4)). These tools facilitate a key subunit disproportionation experiment, which is necessary to establishing the dissociative nature of the proposed quaternary structure equilibrium. This experiment uses hetero-oligomers of PAH and follow methods with which the inventors have had success previously (12).

Second, the invention correlates select disease-associated PAH variants with a shift in the 4mer [symbol] 2mer [symbol] 2mer* [symbol] 4mer* equilibrium. The invention shows that there is a correlation between disease-associated alleles and a shift in this equilibrium using methods as above and approaches successfully applied in the inventors' earlier work (13-14). The inventors' initial focus is on variants in domain-domain interfaces of 4mer* and 4mer models the inventors have prepared, interdomain hinge regions deduced from PAH crystal structures, and the most common disease associated variants (4, 30-32).

Third, the invention uses the derived information to find compounds (morphlocks) that will modulate the equilibrium of PAH morpheein forms (4mer, 2mer, 2mer*, and 4mer*) by binding specifically to one or more of the isoforms (33-37). The invention is related to stabilizing active forms of PAH with small molecules that lead to innovative new conformational therapeutics for deficiencies in PAH activity. Controlling PAH activity through modulation of its quaternary structure equilibrium has the particular advantage of treating a common phenotype for numerous different disease-associated mutations. The invention also relates to applying knowledge on PAH quaternary structure dynamics to develop a diagnostic for this common phenotype. Such a diagnostic discriminates between forms of hyperphenylalaninemia (or PKU) and predicts the clinical response to conformational therapeutics.

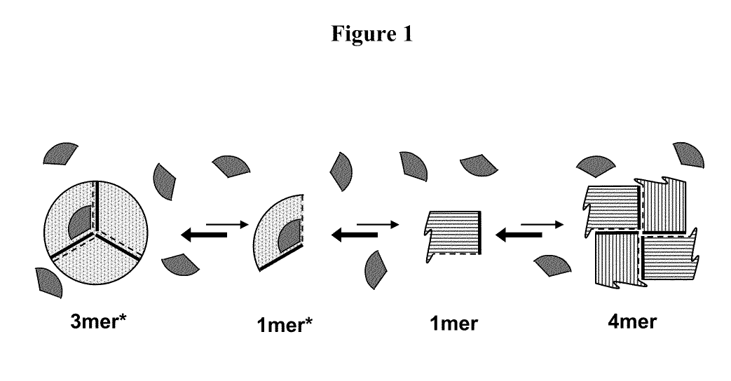

The significance of the invention is two-fold. First is its impact on the development of both novel treatments for the disease PKU and predictive diagnostics for appropriate therapies. Second is the application of the morpheein model of allostery (FIG. 1) to PAH, which sets a precedent for the relevance of this allosteric model to common inborn errors in metabolism. The invention of the morpheein model for the allosteric regulation of PAH stems from an analysis of published PAH data in the context of the inventors' work with PBGS, which is the prototype morpheein (5, 16).

The invention provides that some of the disease-associated alleles encode proteins with an altered quaternary structure dynamic that favors PAH assembly to low activity forms, similar to what the inventors found for all disease-associated alleles of human PBGS (14). The discovery of a novel mechanism for loss of function in PAH opens a valuable new avenue for improved therapeutic intervention and provides tools for improved diagnostics.

The Quaternary Structure Equilibrium of PBGS Correlated with PAH

The inventors' studies of PBGS have uncovered a number of properties that show a morpheein model of allostery (FIG. 1) (16, 19), some of which are enumerated below. The PBGS quaternary structure equilibrium can be described as 8mer [symbol] 2mer [symbol] 2mer* [symbol] 6mer*, where the * indicates a conformational change that results in a different orientation between two domains of each subunit (11-12). Only 8mer has appropriate subunit interfaces to support efficient catalysis (58).

Multiple assemblies—For human PAH, there is strong evidence for an equilibrium of multimers (2, 30, 50, 59-60) most often interpreted as dimers and tetramers, but never before as the 4mer [symbol] 2mer [symbol] 2mer* [symbol] 4mer* morpheein model. A comprehensive treatise on the behavior of full length rat PAH and the response of PAH activity to modulation by Phe, BH₄, lysolecithin, and pH, is consistent with the existence of slowly interconverting and kinetically distinct structural isoforms that follow a more complex relationship than a simple dimer [symbol] tetramer equilibrium (29). Evaluating the PAH literature with the attributes of a morpheein model in mind suggests an equilibrium minimally consisting of high activity tetramers (4mer), high activity dimers (2mer), low activity dimers (2mer*), and low activity tetramers (4mer*), though the precise behaviors of “high” and “low” are not yet strictly defined. As with PBGS, the “*” reflects a posited reorientation between at least two domains of a subunit; this domain-domain reorientation is central to the inventors' morpheein model of PAH. Although the most economical model for the PAH quaternary structure equilibrium is 4mer [symbol] 2mer [symbol] 2mer* [symbol] 4mer*, the inventors examine the data for evidence of larger assemblies, as these have been documented (2-3, 61-62).

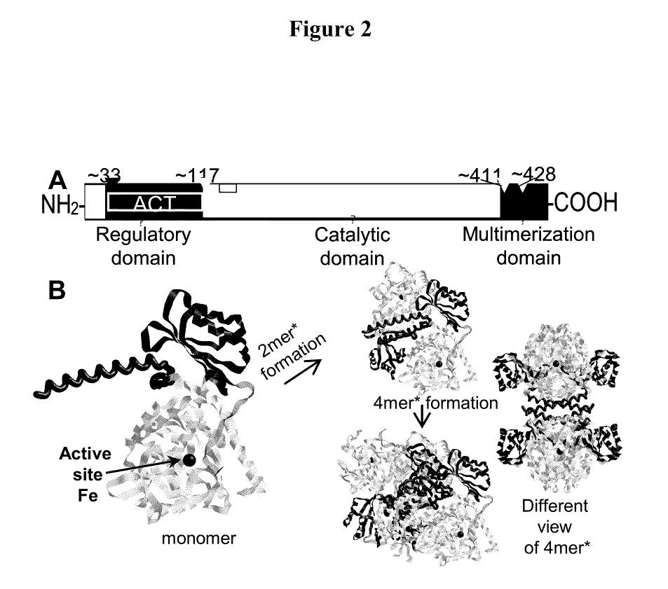

Alternate assemblies modulate access to the active site—In PBGS, the low activity assemblies 2mer, 2mer*, and 6mer* lack a subunit-subunit interaction necessary to stabilize a closed conformation for the active-site lid, which is a mobile loop that gates access for substrate, cofactors, and solvent to the active site (58). Each PAH subunit has three domains (see FIG. 2). The inventors provide that the low activity conformations 2mer* and 4mer* have at least one subunit-subunit interaction that interferes with protein structure dynamics in such a way as to inhibit the normal binding and release of substrates, products, and/or other active site components necessary for catalysis. This is consistent with PAH crystal structure 1 PHZ, wherein a regulatory domain partially blocks access to the enzyme active site (63). Removing this domain results in a constitutively active enzyme.

Kinetic hysteresis—In PBGS, physiologic ligands (e.g. substrate and zinc) at the active site have ligand-protein interactions that stabilize the closed-lid conformation and shift the equilibrium toward 8mer, where this lid conformation is more stable relative to 2mer, 2mer*, and 6mer*. Thus, if PBGS is not all 8mer under a given condition, substrate can activate PBGS by binding at the active site and shifting the quaternary structure equilibrium toward 8mer by stabilizing the closed-lid conformation. This activation is relatively slow and is reflected experimentally as a kinetic hysteresis, where activation occurs over seconds, minutes, or even hours (64). The inventors provide that a similar mechanism for the hysteresis occurs, under select conditions, when Phe is used to initiate a PAH activity assay (29). PAH catalysis requires the substrates Phe and molecular oxygen as well as the cofactors BH₄ and iron, the latter of which appears to remain tightly bound in all quaternary states. Phe and BH₄ activate PAH, and this activation is not additive. Routine enzyme assays often include a preincubation step with Phe. The inventors provide that Phe binding at the active site involves molecular interactions that are more favorable in 4mer and 2mer and less favorable in 4mer* and 2mer*. Thus, Phe binding at the PAH active site (like substrate or zinc binding at the PBGS active site) causes the quaternary structure equilibrium to shift toward the active multimers, which in the case of PAH are 4mer and 2mer. In this model, substrate activation does not require substrate binding at a site other than the active site, but does not dismiss the possible existence of an additional allosteric Phe site in the regulatory domain.

Small molecule modulation of a quaternary structure equilibrium—In PBGS, small molecules can bind to a 6mer*-specific site, stabilize the 6mer* and act as allosteric inhibitors (33-34, 37). In plant and bacterial PBGS, Mg²⁺ can bind to an 8mer specific site, stabilize the 8mer and act as an allosteric activator (11, 65). For PAH, the inventors provide that the existence of a 4mer-specific small-molecule binding site allows for the binding of allosteric activators and the stabilization of the 4mer. New NMR data suggest such an allosteric site for Phe which interacts with a dimeric form of the regulatory subunit (66). In the published model of a PAH tetramer (67), there are no interactions between two regulatory domains; this would require a reorientation of the domains of each PAH subunit. The putative allosteric Phe binding site may correspond to the binding site for the therapeutic large neutral amino acids. Large neutral amino acids may work as morphlocks. In such a case, rational design can be used to optimize the approach.

Quaternary structure equilibrium responds to solvent conditions—In PBGS, alkaline pH and or low ionic strength cause accumulation of 6mer*, presumably due to destabilization of the subunit-subunit interaction that is 8mer-specific (17). Changes in pH and other solvent conditions can change the dimer:tetramer ratio in the quaternary structure equilibrium of PAH; furthermore this is reversible (29).

The key to understanding the kinetics of proteins that function as morpheeins is the appreciation that the quaternary structure equilibrium between active and inactive forms can be in flux during an activity assay, and this flux involves protein dissociation, domain-domain reorientation, and reassociation events. The bulk of the existing data on PAH regulation, even when interpreted with respect to multimerization, has been interpreted in terms of a dimer and a tetramer, but not in terms of a model wherein the activation phenomenon must involve a conformational change in the dimeric, but not the tetrameric, assembly. However, the inventors note that Phe can activate a variant of PAH that cannot form tetramers (29). This observation is consistent with the notion that addition of Phe to the 2mer [symbol] 2mer* equilibrium causes an activating shift toward 2mer. The observation is inconsistent with a more common view of PAH which can be summarized as 2mer* [symbol] 4mer* [symbol] 4mer, wherein there is no dissociation event associated with the activation phenomena. As described in more detail below, the inventors provide that there is a major reorientation of an N-terminal regulatory domain in the activating 2mer* to 2mer transition, that this domain reorientation cannot take place in the tetramer because of steric hindrance in the tetramer, and that the new active conformation is further stabilized by 4-mer specific interactions involving the N-terminal regulatory domains and perhaps also the C-terminal multimerization domain, thus promoting the 2mer to 4mer transition and stabilizing 4mer.

### Correlations Between ALAD Porphyria and PAH Deficiency

ALAD porphyria is a very rare, recessive, inborn error of metabolism caused by alleles encoding PBGS proteins with very low activity. As with PKU, only individuals who are compound heterozygotes with two defective copies of the PBGS gene manifest the disease. Using biochemical and biophysical tools, the inventors have established that each of the eight known ALAD porphyria-associated PBGS variants has a higher propensity to form the 6mer* than wild type PBGS (14). Like PKU, the ALAD porphyria-associated mutations occur all over the protein, indicating that the quaternary structure dynamic responds to small changes in protein structure throughout the protein (14). Mutations associated with low PAH activity constitute the most common inborn error of metabolism. There are ˜500 documented variants, many of which are missense mutations that change a single amino acid (31-32). Reported analyses of PKU-associated variants suggest that some shift the oligomeric equilibrium and/or alter protein stability (2, 30, 50). Disease-associated mutations occur throughout the protein and most are at residues that do not interact directly with the substrates or cofactors. The disease is manifest in compound heterozygotes and the clinical phenotype (as evidenced by increased blood Phe levels) is variable and largely unpredictable (3, 52, 56). The inventors provide that the PAH 4mer [symbol] 2mer [symbol] 2mer* [symbol] 4mer* quaternary structure equilibrium responds to mutations throughout the protein, any one of which can effect the kinetics or thermodynamics of the various transitions (indicated by [symbol] s). This complex equilibrium can explain the highly variable phenotypic manifestations of the disease. The invention provides that small molecule therapeutics that shift the equilibrium toward the active 4mer and 2mer conformations provide an additional therapeutic modality that will afford parents and patients easier maintenance of low blood Phe levels and fewer undesirable consequences in children and adults.

### A New Model for the Allosteric Regulation of PAH

The dynamic nature of proteins contributes to the difficulty seen in acquisition of full length X-ray crystal structures for many multi-domain proteins such as PAH. Combining structural information from different two-domain structures, as has been done to model a three-domain structure for PAH reveals one possible full length structure. The inventors have provided a specific structural hypothesis regarding major domain reorientations, not yet seen in PAH X-ray crystal structures, which must occur in the transition between inactive and active multimers. The inventors have also applied the innovative “protein common interface database” (121), which is drawn from protein assembly data in the PDB, to predict the nature of the required PAH domain-domain reorientation. However, numerous published characteristics of mammalian PAH suggest a dissociative morpheein model for allosteric regulation. Thus, in addition to the active site directed pharmacological chaperone BH₄, which is effective in a subset of patients, the inventors provide a binding surface for a series of allosteric pharmacological chaperones that can also serve as therapeutics for a patients suffering from disease caused by mutations that destabilize the active multimeric conformations. [For pharmacological chaperone, see REFERENCE 93: In pharmacology, the role of a chaperone is similar (to that of a protein chaperone such as the heat shock proteins), but instead of being proteins, pharmacological chaperones are small molecules, and instead of assisting in folding, they usually stabilize an already folded macromolecule (usually a protein) by binding to it and stabilizing it against thermal denaturation and proteolytic degradation. Also Reference 93: Pharmacological chaperones . . . are designed specifically to bind to their target protein and, ideally, stabilize only that macromolecule.] Because active site-directed multimer stabilization is different from allosteric multimer stabilization (using PBGS as a striking example), the population of patients responsive to BH₄ may be either distinct from or overlapping with the population that will be responsive to allosteric pharmacological chaperones.

Application of the morpheein paradigm to the regulation of PAH is a truly innovative way to understand the structural basis of hyperphenylalaninemia and PKU. Although the published literature on PAH reveals numerous features typical of such regulation, a dissociative morpheein allosteric model has yet to be considered and tested. Establishing this model for PAH allows pursuit of pharmacological chaperones, also known as activating morphlocks (33), that promise improved therapeutics for patients with PAH deficiencies. The inventors have delineated predictive criteria for considering whether or not a protein might behave as a morpheein (16, 5) and applied these to PAH as described herein. No one criterion proves the morpheein character of PAH, but all are consistent with the model. Applying a rationale delineated below, the inventors put forth an innovative structural model for allosteric regulation of PAH.

This model is consistent with the published literature on PAH, which reveals numerous features typical of such regulation. The application of this model for PAH allows valuable avenues for the development of improved therapeutics and diagnostics for patients with PAH deficiencies.

The inventors have delineated predictive criteria for considering whether or not a protein might behave as a morpheein, some of which were described above (5, 16). Additional characteristics include the chromatographic or electrophoretic separation of structural isoforms, including alternate multimers. For PBGS, the 8mer and 6mer* can be separated by ion exchange chromatography and/or by native PAGE (11-13, 17). For PAH there is electrophoretic evidence for conformationally distinct tetramers (29) and well documented changes in chromatographic behavior as a consequence of Phe activation (68). These putative morpheein forms of PAH can be demonstrated to interconvert without chemical modification. By definition, different morpheein forms are functionally distinct, such as exhibiting different pH rate profiles (e.g. (11-13)). When morpheein forms are kinetically distinct, kinetic evaluations can show slow hysteresis and steady state data will fit best to a model that considers a mixture of kinetically distinct components (16). The different forms of PAH have different levels of activity and different abilities to be activated by a variety of treatments. None of these PAH properties individually prove the morpheein character of PAH, but they are uniformly consistent with the model. Additionally, published crystal structures for mammalian PAH allow the inventors to put forth an innovative morpheein model for allosteric regulation of PAH that is also consistent with published biochemical data.

The quaternary structure dynamics of PBGS provide a framework for a morpheein model for PAH. PBGS can be described in terms of an N-terminal arm, a central catalytic αβ-barrel, and a C-terminal extension (69). Interconversion between components of the quaternary structure equilibrium involves subunit-subunit association/dissociation and changes in the orientation of one domain of a subunit with another domain of the same subunit. This interconversion involves changes in a small number of backbone Ψ and Φ angles, but does not require gross refolding of the protein. Steric factors prevent the required domain reorientation from occurring in the 8mer or 6mer*. Based on this paradigm, the inventors postulate without being bound to any particular theory that normal allosteric regulation of PAH involves subunit-subunit association/dissociation and changes in the orientation of one domain of a subunit with another domain of the same subunit, with changes in a few backbone angles at hinge regions, but not protein unfolding/refolding. Thus, the morpheein model of PKU as a conformational disease is distinct from views that consider disease-associated PAH variants as defective in protein folding (3, 6-7). In the current morpheein model, the defect is in the kinetics and thermodynamics of the transitions ( [symbol] ) that govern the allosteric quaternary structure equilibrium (e.g. 4mer [symbol] 2mer [symbol] 2mer* [symbol] 4mer*). The inventors' hypothesis is supported by the observation that activation of PAH causes a change in a dimer-tetramer ratio, but does not cause changes in indices of protein folding (e.g. CD spectra) (29).

### Rationale

The structure(s) of mammalian PAH lead to a morpheein hypothesis for the regulation of PAH function. Mammalian PAH is composed of three domains (FIG. 2A). The residues required for catalysis lie in a catalytic domain (˜residues 118-410). Much of the mechanistic work on PAH has been done on a catalytic fragment (residues ˜103-428) (e.g. (21, 70-71)), and many structures have been solved for this fragment with various ligands defining the active site (72-76). PAH contains a regulatory domain (˜residues 1-117); the N-terminal 33 amino acids are autoregulatory and partially block the active site in crystal structures containing only the regulatory and catalytic domains of rat PAH (63). The rest of the regulatory domain is an ACT domain, which is generally implicated in regulation of metabolic enzymes (77). The published report on the rat PAH two-domain structures notes the highly hydrated and hydrophilic nature of the interface between the catalytic and regulatory domains and suggests that this interface may be transient. The transient interface in the PBGS quaternary structure equilibrium is also highly hydrated and hydrophilic (17) and the inventors have delineated this as a general characteristic of soluble proteins that use the morpheein model of allosteric regulation (16). The final domain of human PAH is a multimerization domain (˜residues 411-452), which can be divided into a dimerization segment (˜residues 411-428) and a tetramerization segment (residues 429-452). A two-domain human PAH structure containing the catalytic and multimerization domains (residues 118-452) is a tetramer; the tetramerization segment is α-helical and forms a domain swapped 4-helix bundle. Consistent with full-length PAH existing as an equilibrium of multimeric forms with major domain reorientations, a crystal structure of full-length PAH has not yet been solved (1). As described below, FIG. 2B draws on the rat and human PAH two-domain structures to model a human PAH tetramer.

The following key published observations contribute to the basis of the PAH allostery model:

The catalytic fragment has the same specific activity as the full length protein after activation by Phe (˜1 mM) (50). Therefore, the inventors' models of the low-activity protein multimers include the regulatory domain limiting access to the PAH active site (as is seen in the two-domain rat PAH crystal structure). The current model of the high-activity conformation involves a significant reorientation of the entire regulatory domain that relieves the ability of the regulatory domain to modulate active site access.

Activation by Phe causes a shift in an equilibrium of PAH dimers and tetramers to favor the tetramer (29). The inventors' model for a high-activity tetramer contains a tetramer-specific subunit interface that can bind allosteric Phe molecule(s). This subunit interface is seen broadly as the target for small molecule therapeutic chaperones designed to stabilize the active tetramer. Because of the toxicity of Phe, such therapeutic chaperones are not proposed to be Phe-mimics, but rather may bind anywhere on this oligomer-specific subunit interface.

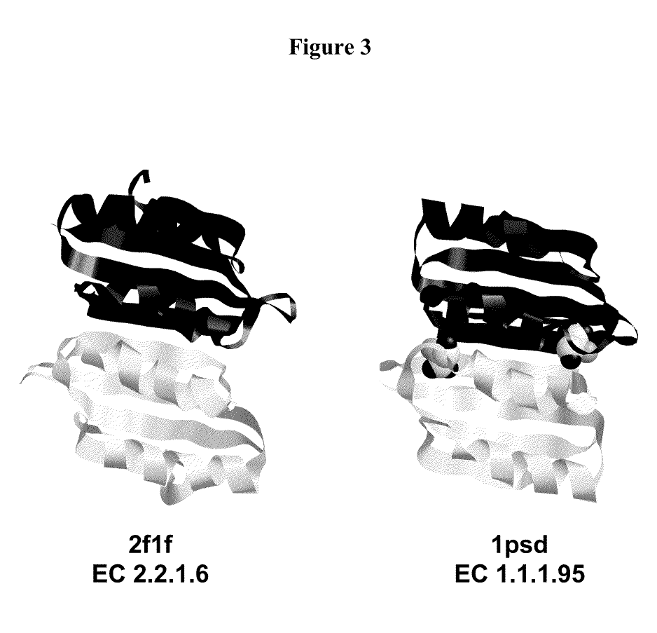

The transient formation of an ACT-ACT interface that can be stabilized by binding Phe allosterically is a key component of the inventors' hypothesis concerning the structure of the Phe activated PAH tetramer. ACT domains, such as the one that constitutes the bulk of the regulatory domain of PAH (FIG. 2A), are found in diverse proteins involved in amino acid or purine synthesis, many of which are allosteric and respond to the allosteric binding of amino acids (77, 122). Except for PAH, all of the ACT domains found in the PDB are involved in formation of intersubunit ACT-ACT dimers. As illustrated in FIG. 3, some of these contain amino acids at the ACT-ACT multimerization interface while others do not. ACT-ACT dimers are not seen in the PAH crystal structure nor in the three-domain PAH tetramer model (FIG. 2B). The inventors' analysis of crystal packing in the rat PAH structure also does not reveal cryptic interactions between ACT domains. Supported by the diverse stoichiometry of ligand binding to ACT-ACT interfaces (see FIG. 3), this model is consistent with experimental data indicating Phe binding to a PAH allosteric site at both 0.5 per subunit and 1 per subunit (29); both may be correct depending on the conditions of the experiment (as the inventors have found with the stoichiometry of ligand binding to PBGS) (123). Allosteric Phe binding to bifunctional E. coli chorismate mutase-prephenate dehydratase (CM-PD) provides a strong precedent for the inventors' view of how Phe promotes tetramerization of PAH by binding to its ACT domain. CM-PD is a three-domain protein that contains an ACT domain involved in negative allosteric activation by binding Phe (124-125). Although a crystal structure of full length CM-PD is not available, it is known that Phe binding to the ACT domain causes the dimeric protein to form a low-activity tetramer (126).

Using the most advanced tools available (78-82) and the FCCC Molecular Modeling Facility, the inventors have combined the available crystallographic information to create a model of the conformation of a full length human PAH tetramer (FIG. 2B). This is a model for the low activity 4mer* with the N-terminal autoregulatory sequence partially blocking free access to the enzyme active site. Consistent with full-length PAH existing as an equilibrium of morpheein forms that involve major domain reorientations, no crystal structure of the full-length protein has yet been solved (1).

### PAH Morpheein Model

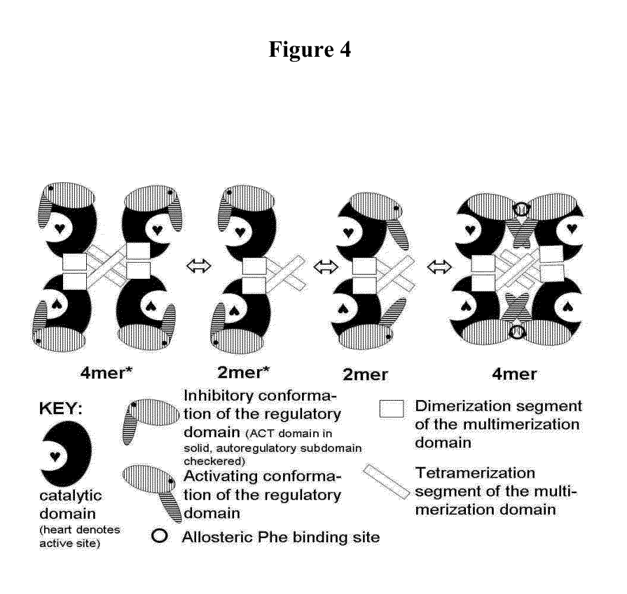

A schematic morpheein model for allosteric regulation of PAH is presented in FIG. 4; a key element of this model is that the proposed spatial reorganization of the regulatory domain relative to the catalytic domain cannot occur in the tetrameric forms. This model is fully consistent with experimental mutation data that assign exposure of Trp120 as the source of the fluorescence change that accompanies PAH activation (27); Trp120 is not exposed in the inventors' modeled 4mer*, but would become exposed by the rearrangement of the regulatory domain as proposed in FIG. 4. In addition, the lysine-rich tryptic digestion site is solvent-accessible in a hinge region between the regulatory and catalytic domains of 4mer*. This site would become buried in the activating conformation of the regulatory domain illustrated schematically in FIG. 4.

In this model, 4mer and 2mer are active conformations that can appropriately modulate ligand access to the enzyme active site, while 2mer* and 4mer* include a conformation of the regulatory domain that blocks (or partially blocks) entry and exit of active site components (see FIG. 2B). The inventors provide that the active 4mer is a fragile assembly whose stability is compromised by many different PKU-associated PAH variants, similar to what the inventors have established for ALAD porphyria-associated PBGS variants (14). Furthermore, small molecules that can bind to a 4mer-specific site can stabilize the 4mer and act as a therapeutic for PKU and hyperphenylalaninemia. One such site, which is proposed to exist at an ACT-ACT interface, may be the binding site for an allosteric Phe (see FIG. 4) (26, 50). In a published PAH tetramer model ((67) & FIG. 2B), there are no interactions between two regulatory domains; this would require a reorientation of the domains of each PAH subunit (see the 2mer* [symbol] 2mer portion of FIG. 4).

Recent biophysical data support the notion that Phe can bind to and alter the conformation of a dimeric assembly of the PAH regulatory domain (66). This important study supports early work by Shiman which showed Phe binding to rat PAH at a location where it could not be hydroxylated but could activate the enzyme (26). Another recent study using hydrogen/deuterium exchange to monitor Phe interactions with full length rat PAH is fully consistent with the proposed dissociative model (127). This H/D exchange work supports a dramatic change in the interactions between the regulatory and catalytic domains. Unfortunately, that study could not address the solvent accessibility of the PAH multimerization domains because the peptides of the multimerization domain could not be identified.

The model holds that PAH activity is governed by an equilibrium of quaternary isoforms according to the morpheein model of allosteric regulation. The key to establishing that PAH is a morpheein is to demonstrate that there are two different kinds of tetramers and that their slow interchange requires a dissociation step. The latter can be established by demonstrating chain disproportionation during the transition between 4mer* and 4mer. In general the inventors apply approaches the inventors have pioneered to study the quaternary structure equilibrium of PBGS (11-15, 17).

1A—Developing the necessary tools.

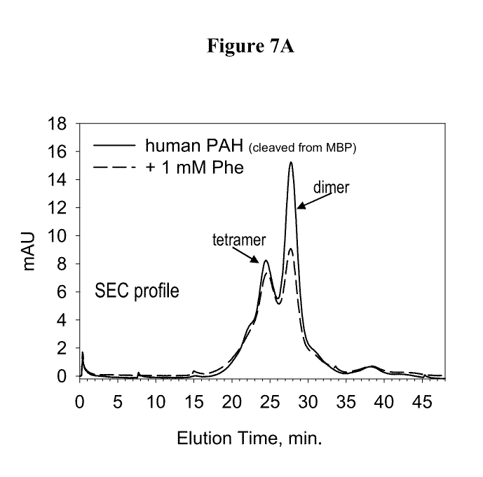

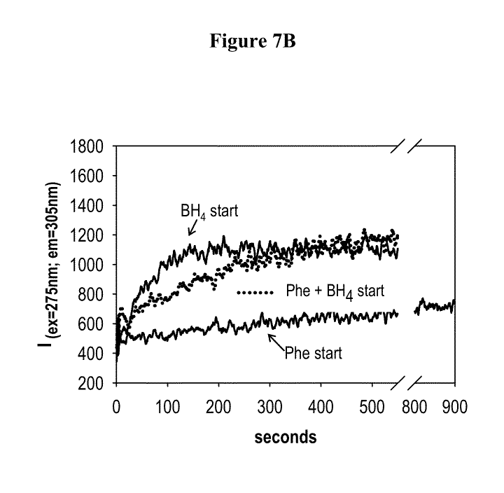

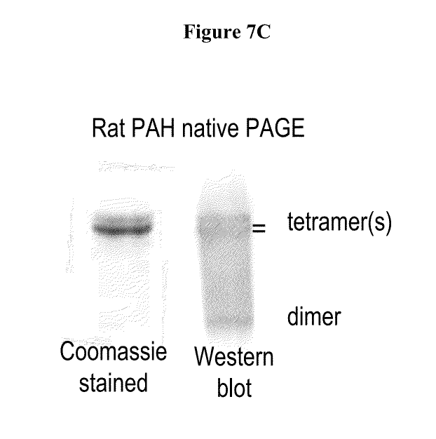

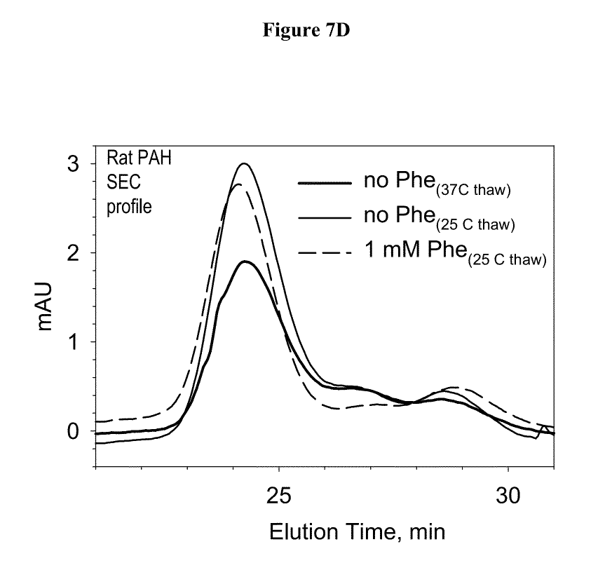

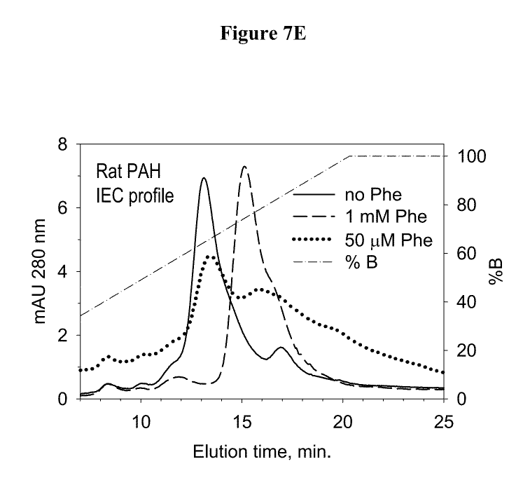

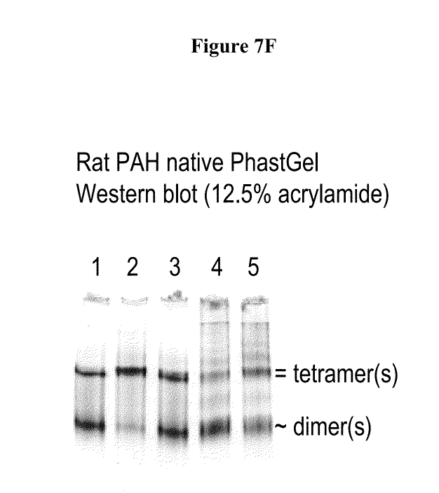

1Ai—Protein expression and purification—Recombinant full length human PAH can be overexpressed in E. coli to high levels with or without cleavable (N-terminal) purification tags (e.g. (25, 27, 51, 60, 83)). The native (not tagged) proteins are expressed and purified to chromatographically separate isoforms. Low-activity form(s) can be separated from the Phe-activated forms using IEC (see FIG. 7E). Initially, the inventors draw on an affinity method pioneered by Shiman (68), wherein expressed PAH is preincubated with Phe to convert the protein to the active form(s) which is adsorbed onto a hydrophobic column, and eluted in buffer that does not contain Phe (presumably in the low activity form). This method takes advantage of the major conformational change induced by the substrate Phe, and which can be readily monitored by established methods described below (68). This major conformational change corresponds to a transition between a predominantly 2mer*/4mer* mixture to a predominantly 2mer/4mer mixture (see FIG. 3). Ionic strength and pH effects for quantitative elution of the PAH in the various forms are investigated. With this partially purified protein (expected to be >90% PAH), chromatographic behavior of the native morpheein forms on ion exchange and size exclusion columns can be linked to factors that have been documented to modulate the kinetic behavior of PAH (e.g. Phe, BH₄, pH) (see FIG. 7), and thus are attributed to modulating the equilibrium of morpheein isoforms. The major conformational change facilitates the separation of 4mer, 2mer, 2mer*, and 4mer* using ion exchange chromatography as has been successfully applied to separation of 8mer, timer*, and alternate dimers of human PBGS (11-14). For isolated pools, protein size distributions are determined using size exclusion chromatography (SEC), native PAGE, or blue native PAGE, all of which can readily discriminate between dimers and tetramers. The techniques used to discriminate between different conformational states of PAH are described below (4). Of note is the propensity of oligomers in a given chromatographically separated sample to slowly drift back to an equilibrium distribution (e.g. (13)). Temperature, pH, protein concentration, and buffer components can modulate the position of the quaternary structure equilibrium and the rate of reequilibration.

1Aii—Application of methods that discriminate between PAH morpheein forms.

Limited Tryptic Digestion

Limited tryptic digestion, analyzed by SDS PAGE, is a straightforward conformational probe. In the inventors' model of 4mer*, the hinge region 111-117 is solvent exposed, while it is buried in the 4mer model. This lysine rich sequence RDKKKDT is particularly susceptible to tryptic digestion. Consequently, the extent of tryptic digestion under a fixed set of conditions is a straightforward estimate of the percent of the protein sample in the 2mer* [symbol] 4mer* portion of the equilibrium. This tool coupled with native PAGE analysis and/or analytical SEC provides appropriate diagnostics to discriminate between 4mer, 2mer, 2mer*, and 4mer*.

Kinetic Characterization

The following techniques are used: Comparative assays with and without preincubation with Phe or BH₄, time course assays that evaluate hysteretic behavior, and evaluation of rate vs. Phe concentration for hyperbolic or sigmoidal behavior (28). Fractions rich in 4mer and/or 2mer are active and insensitive to activation by Phe. Fractions rich in 4mer* or 2mer* are of lower activity, sensitive to activation by preincubation with Phe, or show kinetic hysteresis (slow activation on the time scale of minutes) when assayed with Phe in the absence of preincubation. For PBGS the inventors have taken advantage of differences in the pH rate profiles of the quaternary structure isoforms. Such features are a useful diagnostic tool for PAH.

Native PAGE

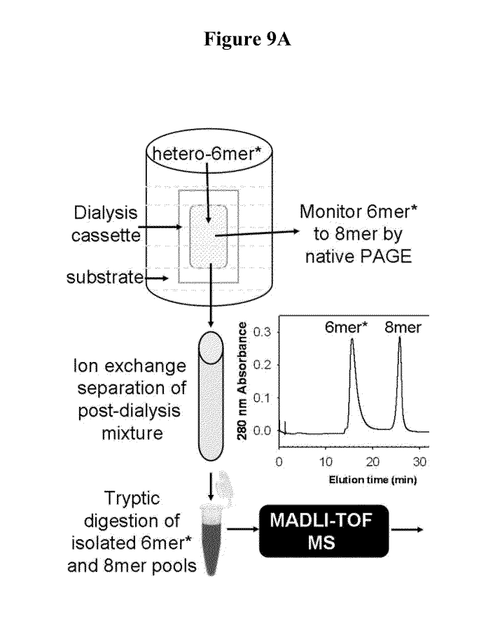

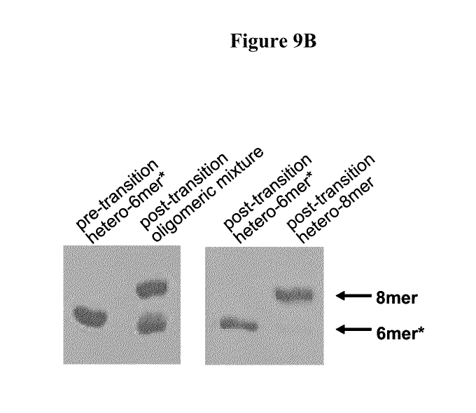

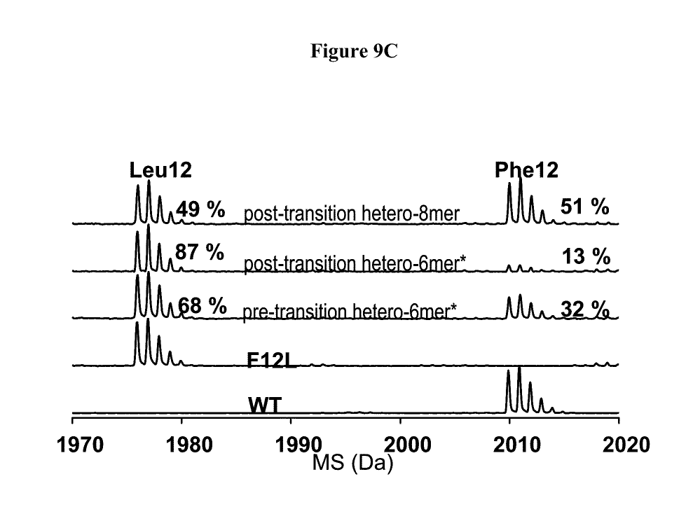

The major conformational change between nmer and nmer* allows the separation of the components using native PAGE, which has been an invaluable tool for investigating factors that affect the quaternary structure equilibrium of PBGS (e.g. FIG. 9). Early work with mammalian PAH showed closely spaced bands on native PAGE which the inventors believe correspond to separation of 4mer and 4mer* (29) (see also preliminary results presented in FIG. 7). The inventors have demonstrated that both native PAGE and ion exchange chromatography can serve to separate different metastable conformations of the human PBGS variant W19A, which is an obligate dimer, but which can be separated into two different dimers (13).

Alternate Biophysical Tools

Flatmark and coworkers have used complementary conformational probes to investigate conformational transitions in PAH, which can be used to assign the fractionated pools to components of the proposed 4mer [symbol] 2mer [symbol] 2mer* [symbol] 4mer* equilibrium (4). These are surface plasmon resonance, tryptophan fluorescence, and circular dichroism monitored thermal denaturation. Different components of the quaternary structure equilibrium display different behaviors when subjected to these analytical tools.

1B—Subunit Disproportionation

Establishing the dissociative nature of the transition between PBGS 8mer and 6mer* relied on the preparation of mixed oligomers of the wild type protein (predominantly 8mer) and the naturally occurring F12L variant (predominantly 6mer*) (11-12). Using a bicistronic tandem expression system, the inventors prepared mixed heteromers containing chains with both Phe12 and Leu12. The hetero-8mers and hetero-6mer*s were separated by ion exchange chromatography and analyzed by mass spectroscopy to determine the mole fraction of Phe12 and Leu12 in each hetero-oligomer. As illustrated in FIG. 9, the isolated hetero-6mer* was then dialyzed against assay buffer containing substrate, which favors formation of 8mer. After partial conversion of hetero-6mer* to hetero-8mer, the protein was removed from the dialysis chamber and the resulting hetero-8mer and hetero-6mer were again separated using ion exchange chromatography. These pools were reanalyzed for the mole fractions of Phe12 and Leu12 containing chains. The resulting 8mer was enriched in Phe12, while the remaining 6mer* was enriched in Leu12, thus showing that the individual chains had disproportionated and unequivocally establishing the dissociative nature of the interchange between 6mer* and 8mer (12).

The key to repeating this experiment for PAH is to identify a PAH variant that has a high propensity to exist as 4mer* under conditions where the wild type protein exists mostly as 4mer. Fortunately, it is established that bicistronic tandem expression of variant PAH proteins results in hetero-oligomers (83). Many human PAH variants have been described in the literature which might favor 4mer*. Some mutations are at positions that lie at hinge regions, which are the likely locations of backbone twist in the interdomain reorganization 2mer [symbol] 2mer* transition (see FIG. 3). One variant is T427P, which has been shown to dramatically shift the dimer-tetramer equilibrium, and which does not show the kinetic cooperativity typically seen with respect to the Phe substrate (4). In fact, a general approach is to limit rotation at hinges in the regulatory domain (e.g. Thr117 or Gly33) by mutation to proline. Alternatively, the inventors can follow a lead from PBGS wherein the inventors dramatically shifted the 8mer/6mer* equilibrium by removing an arginine at the dissociable hydrated/hydrophilic interface. In PAH, Arg261 (where R261Q is disease-associated) and Arg243 (where R243Q is disease-associated) are at the interface between the catalytic domain and the regulatory domains.

Correlating Select Disease-Associated PAH Variants with a Shift in Equilibrium

Methods and rationales successfully used to correlate disease-associated PBGS variants with alteration of the quaternary structure equilibrium (14) are followed to associate PAH quaternary structure equilibrium and variants with hyperphenylalaninemia and PKU.

Disease-associated PAH variants are expressed without tags and analyzed using methods described above. Proportions of the various oligomers are determined by chromatographic separation of isoforms during purification. The kinetic, biochemical, and biophysical properties of the resulting separated fractions are correlated with alternate multimers as described above. A relationship between disease-associated variants and a shift in the mole fractions of 4mer and 4mer* is established. The inventors provide disease-associated variants in domain-domain interactions of the 4mer* model, the 4mer model, interdomain hinge regions, and the ten most common disease associated variants (4, 30-32), including R408W, IVS10-11G>A, I65T, R261Q, P281L, IVS12+1G>A, R158Q, E280K, Q232Q, Y414C, R252W, V245V, and L48S (31-32). This demonstration allows the discovery of 4mer-stabilizing allosteric effector molecules.

Modulator Compound (a.k.a. Morphlock) Identification

The multimeric protein modulator compound can bind anywhere to stabilize the active forms of PAH, but the binding site must stabilize one quaternary structure. Binding is preferable to a site that is present in one morpheein form but not the other. The identification methods of such compounds are similar to those used in the identification of PBGS inhibitors described in U.S. Pat. No. 7,863,029, incorporated herein. More specifically, these quaternary structure modulators (a.k.a. morphlocks) were discovered using a combination of in silico docking followed by in vitro testing (34, 35-36) and also by a direct native PAGE mobility shift screen of the Johns Hopkins Clinical Compound library (JHCCL) and the National Toxicology Program (NTP) environmental contaminants library (37, 142). In all cases compounds that can shift the PBGS equilibrium to favor low-activity assemblies were identified and in most cases it was established that this oligomer stabilization caused inhibition of enzyme activity. In the case of human PBGS, the inventors established that a handful of drugs stabilize the 6mer* and inhibit activity, that these inhibitors were more potent when tested with the ALAD porphyria-associated variants, and that some of these compounds had been reported to cause porphyria-like side effects (37, 34). Of the ˜1500 compounds in the JHCCL, 12 drugs stabilize the human PBGS hexamer and inhibit human PBGS activity in vitro. In vivo drug inhibition of human PBGS through hexamer formation would represent an unprecedented mechanism for drug side effects. The inventors considered small-molecule perturbation of quaternary structure equilibria as a general mechanism for drug action and side effects. Human PAH is a specific example of the perturbation of quaternary structure equilibria by a drug. Although the long term goal of the inventors' work with PAH is to identify pharmacological chaperones that act to stabilize the active conformations 4mer (and perhaps also 2mer), compounds that stabilize the inactive conformations are also provided. These drugs could be contraindicated in PKU and hyperphenylalaninemia patients.

The inventors provide a native PAGE mobility shift screen of the JHCCL and NTP libraries against human PAH, following procedures established for PBGS (37, 142) by the inventors. Because human PAH exists as an equilibrium containing substantial amounts of both tetramer and dimer, the initial screen looks for compounds that can perturb this ratio (as demonstrate for Phe in FIG. 7A). Human PAH has a higher propensity to exist as a dimer, relative to the rat protein. This propensity contributes to its reputation as the “more difficult” or “less stable” of the mammalian PAHs. Although it is straightforward to monitor the dimer:tetramer ratio, it may also prove possible to monitor the ratio of the two closely spaced tetrameric bands that are two architecturally different tetramers (4mer and 4mer*) (FIG. 7C).

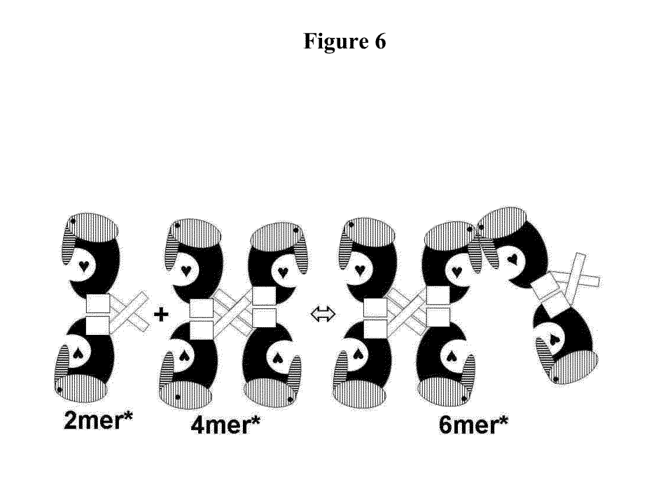

The quaternary structure modulators that favor a specific oligomer are tested to determine whether these compounds can modulate the activity of PAH (activation and/or inhibition), and to quantify the potency of these putative allosteric effectors as the inventors have done before (37). Together the two cited libraries contain ˜2900 compounds. A compound collection of such a size can be manually screened, in duplicate using the PhastGel technology (two instruments). The invention is guided by the knowledge that the PAH quaternary structure equilibrium is sufficiently fragile to respond to changes pH, temperature, and ionic strength. Small molecules structurally unrelated to Phe that can modulate the human PAH quaternary structure equilibrium are identified with one caveat that it must be insured that small molecule stabilization of the ACT-ACT interface is selective for 4mer stabilization and does not promote aggregation as illustrated in FIG. 6.

Modulators of the invention using a similar in silico screening approach provided that a crystal structure or model structure can be prepared for the active quaternary isoforms of the target protein.

The in silico approach is an approach to drug discovery. The in silico approach has three components. These are the small molecule virtual libraries, the protein structure models (or protein crystal structure models) to which these libraries are docked, and the computational docking software. The development of software for virtual library screening by docking small molecule structures to protein structures is being intensely developed by many groups [cf. 84]. Programs at present are estimated to provide at least a 50-fold increase in the probability of finding a ligand compared to random screening, although the inventors' experience has been much better. An exemplary docking program is the commercially available program Glide (Schrödinger Inc., 2003).

### 1. Building the in Silico Library of Compounds for Docking

The computational docking studies require preparation of the in silico libraries of small molecules and the docking using Glide. The compounds available from Life Chemicals, Inc. are predominantly molecules of molecular mass ˜500 Daltons, and are designed to be relatively “drug-like” (e.g. adhering to Lipinski's rule of five). Two dimensional representations of compounds available from Life Chemicals, Inc. were obtained in SD format from the vendor. Scripts supplied by Schrödinger Inc. were used to convert these into three dimensional, energy minimized representations in a file format used by Glide. In this process the structures were converted to Maestro format, and entries that contained metal ions or atom types other than H, C, N, O, P, S and halogens were discarded. Hydrogens were then added to all atoms as appropriate for the structures, generating a single stereoisomer per compound. The structures were energy minimized using MacroModel with the MMFFs force field and then expanded to include all forms likely to be present in the pH range 5-9 using Schrödinger's Ionizer utility. The structures were again energy minimized using MacroModel, which yielded output files that were suitable for docking with Glide.

### 2. Docking

Docking studies use Life Chemical, Inc.'s compound libraries, which are comprised of small molecules (molecular weight ˜500) that are designed to be relatively “drug-like” (e.g. adhering to Lipinski's Rule of Five [Lipinski, 2001]). Two libraries, named “G-protein coupled receptor-targeted” and “Kinase-targeted” are used. Proposed binding sites include subunit interfaces and/or surface cavities that are specific to one of the PAH oligomers, such as 4mer. One region of interest is the surface and/or binding sites created by the formation of the PAH ACT:ACT intersubunit dimer. Using this surface, one or more boxes are defined to be used by Glide as the search region within which each entire docked molecule must fit in order to be scored. In the past, we have used boxes 25 Å in dimension. A second smaller concentric cubic region with dimension on the order of ˜14A is used to restrict the location of the center of each docked molecule. The docking process uses the default settings of Glide (version 3.5) Standard Precision (SP) mode and produces a “Glide score” for each molecule; the Glide score is based on a proprietary modification of the chemscore algorithmthat quantitatively accounts for characteristics of the binding interaction between each docked small molecule and the protein. The Glide SP mode scores goodness of fit based predominantly on geometries. For completed studies for each library and each binding site, the inventors found that the Glide scores of approximately 80% of the docked compounds similar to each other; 10% are significantly lower (better fit), and 10% are significantly higher (inferior fit). Compounds with the best SP Glide scores, ˜10% for each library, are then docked a second time using the Extra Precision (XP) mode of Glide, resulting in a set of new, quantitatively unrelated Glide scores. XP mode scoring is more rigorous as it takes into account polarity and hydrophobicity, and penalizes mismatches of hydrophobic-hydrophilic contacts or charges. Again, roughly 80% of the molecules are expected to have similar scores. Molecules with XP Glide scores in the top ˜10% (about 1% of the starting library) are then analyzed further to select for predicted solubility, interactions with the selected binding sites, and variety in both chemical structure and binding interactions. This results in the selection of a set of ˜100 diverse, putatively soluble, small molecules for purchase from Life Chemicals, Inc and in vitro testing.

### 3. Post-Docking Processing

Molecules with XP Glide scores in the top ˜10% (about 1% of the starting library, 3500 compounds) are then analyzed further to select compounds. Compounds may be selected for testing based on, for example, the following criteria. Each selected molecule must make van der Waals contacts or hydrogen bonds with two PAH subunits. It is preferred that each selected compound must have a predicted solubility (Log S) estimate of at least −6, which was calculated using QikProp. The set of selected molecules will preferentially contain a broad sampling of dissimilar structures. The set of selected molecules must represent diverse positions within the docking box.

### 4. High Throughput Screening

A complementary approach to the inventors' computational docking is high throughput screening (HTS). This approach has the potential to identify morphlocks that bind to sites other than those the inventors have defined for in silico docking. “High throughput screening” (HTS) refers to a plurality of assays that test a plurality of compounds, performed robotically, the results of which are generally measured electronically by changes in magnitude or wavelength maxima of absorption or emission of light for the purpose of finding a drug candidate (“hit”) among the compounds. In general, the assay measures an enzyme activity; and cleavage of a labeled substrate to a product causes a change in color, or wavelength of emission, or extent of emission, of such that multiple parallel samples can be read automatically. In general, multiwell plastic plates having at least 96 wells per plate, or 384 wells/plate, or 1536 wells/plate, are used in HTS. Because HTS is highly automated, it is generally performed on at least a plurality of compounds, for example, at least 1,000 compounds, for example, at least 2,000 compounds, at least 5,000 compounds, or at least 10,000 compounds. Libraries of compounds can be obtained, for example, from commercial sources such as ChemBridge (San Diego, Calif.), or Life Chemicals, Inc. For evaluation of the effect of an agent on PAH quaternary structure distribution, Light Scattering is an attractive method of detection and the technology is available in a plate reader format.

### 5. Medium Throughput Native Page Mobility Shift Screen

Small molecule compound libraries of 1500 compounds are screened in duplicate using native PAGE. This medium throughput native PAGE mobility shift approach allows us to complete the screen rapidly using PhastGel technology.

Each of the compounds is subject to an initial screen where each sample contained 2 μL of a 10 mM solution of compound in DMSO or ddH₂O. Samples are prepared by mixing PAH [(0.3 mg mL⁻¹) in an appropriate buffer (e.g. 0.1M Bis-Tris propane-HCl, pH 8.0 (8 μL) with 10 mM compound in DMSO or H₂O (2 μL). The resultant samples, which contained 20% DMSO and 2 mM compound, are incubated for a fixed time at a fixed temperature, before loading and running the gels in duplicate. Electrophoresis is performed using a PhastSystem with PhastGel native buffer strips, and 6-lane (4 μL per lane) applicators are used to load the samples. Separations are performed using polyacrylamide gels and each gel contains a negative control (incubation with DMSO alone) and a positive control. After separation, gels are developed on the PhastSystem using Coomassie Blue stain. The native gel mobility shift evaluation is repeated for each of the preliminary hits using freshly purchased stocks prepared at 10 mM in DMSO unless otherwise noted.

The compounds confirmed to stabilize the PAH 4mer are further examined by native PAGE as a function of compound concentration. Samples are prepared as described above, but with varying concentration of each compound (e.g. 0, 30 μM, 100 μM, 300 μM, 1 mM, and 2 mM). The final concentration of DMSO in each sample is maintained at 20%.

Quantification of PAGE results by densitometry is carried out using the program ImageJ. Three separate determinations are made to quantify the density of each gel band.

Test Compounds

Test agents that can be screened with methods of the present invention include polypeptides, β-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, small molecules, siNA, siRNA, dsRNA, dsDNA, anti-senseDNA, nucleic acids, antibodies, polyclonal antibodies, monoclonal antibodies, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be natural occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of morpheeins forms, including, for example, PAH. A number of assays are available for such screening.

Libraries of test compounds to be screened with the claimed methods can also be generated based on structural studies of morpheeins Such structural studies allow the identification of test compounds that are more likely to bind to morpheeins. The three-dimensional structure of morpheeins can be studied in a number of ways, e.g., crystal structure and molecular modeling. Methods of studying protein structures using x-ray crystallography are well known in the literature. See Physical Bio-chemistry. Computer modeling of a target protein (e.g., a morpheein) provides another means for designing test compounds for screening modulators of the target protein. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR).

Modulators of the present invention also include antibodies that specifically bind to one morpheein form. Such antibodies can be monoclonal or polyclonal. Such antibodies can be generated using methods well known in the art. For example, the production of non-human monoclonal antibodies, e.g., murine or rat, can be accomplished by, for example, immunizing the animal with morpheeins or its fragment. Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to morpheeins.

Human antibodies against morpheeins can also be produced from non-human transgenic mammals having transgenes encoding at least a segment of the human immunoglobulin locus and an inactivated endogenous immunoglobulin locus. See, e.g., Lonberg et al., WO93/12227; Kucherlapati, WO 91/10741. Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using morpheeins.

### EXAMPLES

### Example 1

### Building the Low-Activity 4Mer* Model

Using the most advanced tools available to the inventors (78-82), the inventors have combined the available crystallographic information on two-domain rat and human PAH structures to create a model of one possible conformation of a full length human PAH tetramer (FIG. 2B). The regulatory domain of this model is a homology model of the human PAH regulatory domain (residues 19-117) based on the rat PAH regulatory domain (PDBid 1PHZ). A similar full length human PAH model has been published (67) This is a plausible model for a low-activity 4mer* with the N-terminal autoregulatory subdomain partially blocking free access to the enzyme active site. One half of this structure, maintained by the dimerization segment of the multimerization domain is a reasonable model for a low-activity 2mer*, where the autoregulatory domain continues to limit active site access. BH4 has been shown to bind significantly more tightly to the low-activity form(s) of PAH (Kd ˜0.1 μM) relative to the high-activity forms (Kd ˜14 μM) (128), suggesting that BH4 draws the equilibrium toward the 2mer*/4mer* containing portion of the equilibrium. Stabilization of these lower activity forms, perhaps by bridging the active site and the autoregulatory subdomain, would explain the observation that BH4 inhibits PAH activity (see FIG. 7B).

### Example 2

### Building the High-Activity 4 Mer Model

In construction of the model for active conformations of PAH, in which the active site is not blocked by the regulatory domain, the inventors have drawn upon the observation that ACT domains are often involved in a dimerization interface (see FIG. 3). The published report on the rat PAH two-domain structures notes the highly hydrated and hydrophilic nature of the interface between the catalytic and regulatory domains and suggests that this interface may be transient (30), which is consistent with a reorientation between the regulatory and catalytic PAH domains and consistent with the H/D exchange studies (127). The transient interface in the PBGS quaternary structure equilibrium is also highly hydrated and hydrophilic (17); the inventors have delineated this as a general characteristic of soluble proteins that use the morpheein model of allosteric regulation (16).

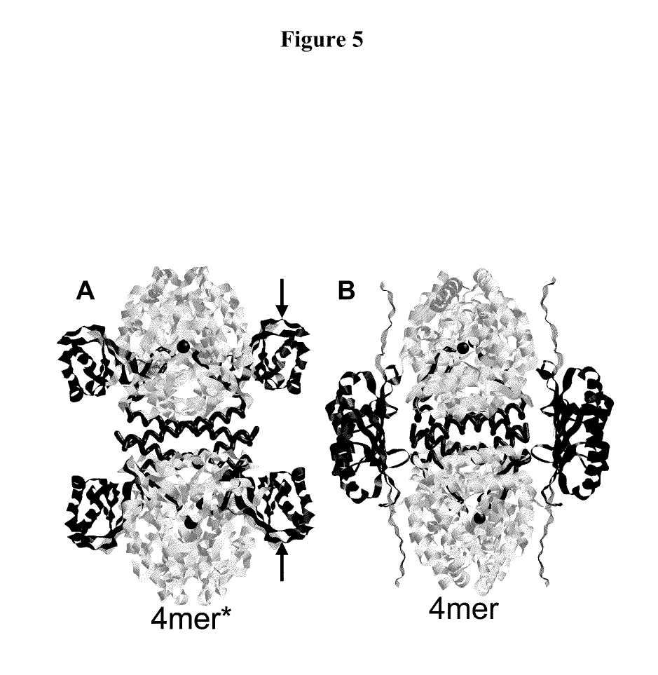

The inventors provide that the structure of the active 4mer can be approximated by drawing on the ACT domain dimers in the PDB (FIG. 3). To form a homologous ACT domain dimer from the 4mer* model illustrated in FIG. 2B requires dissociating the interface between the regulatory domain and the catalytic domain, and twisting each of the regulatory domains by approximately 180°. The protein surfaces that are required to come together in an ACT dimer are pointing outward in the 2mer* and 4mer* models (FIG. 5A). If these surfaces come together when PAH is in the low-activity multimers, the reported existence of larger PAH multimers (see FIG. 6, e.g. (86)) can be explained.

The inventors have prepared a preliminary 4mer model (FIG. 5) as follows: 1) Start with the homology model of the human PAH regulatory domain described above. 2) Dimers of the regulatory domain were constructed by orienting the ACT portions of the regulatory domain according to the geometric relationship present in the ACT domain dimer of E. coli phosphoglycerate dehydrogenase (PDBid 2P9C, FIG. 3) to create a model of the human PAH regulatory domain dimer with subsequent refinement of side chain rotamers in this dimer using SCRWL4 (79); 3) The regulatory domain dimer models were centered on both sides of the human PAH two-domain tetramer structure (residues 118-452, PDBid 2PAH). 4) Connection of the regulatory and catalytic domains occurred at residue 117, around which loop modeling and energy minimization was performed using steepest descent methods (123). The inventors provide methods for modeling the N-terminal 33 residues, which are not predicted to be disordered (124). The inventors provide the preparation a family of possible 4mer models using Rosetta to dock this region to nearby portions of the protein structure model, including portions of the multimerization domain.

The tetramer models shown in FIG. 5 are fully consistent with published data on changes that accompany Phe activation of PAH. 1) Experimental mutation data assigned Trp120 as the source of the fluorescence change that accompanies PAH activation (27); Trp120 is not solvent exposed in the modeled 4mer*, but is exposed in the modeled 4mer. 2) PAH has a lysine-rich tryptic digestion site between the regulatory and catalytic domains (4). This site is exposed in 4mer* and buried in 4mer.

### Example 3

### Protein Expression/Purification and Characterization

The inventors have obtained expression vectors for full length untagged rat and human PAH from Prof. P. Fitzpatrick and an expression vector for an N-terminal MBP-fusion for human PAH from A. Martinez. The inventors have found in preliminary studies with the tagged human PAH, as the inventors expected, that the tag must be removed prior to any meaningful studies on the PAH quaternary structure equilibrium. Unfortunately, proteolytic cleavage of the MBP tag cannot be quantitatively achieved without some proteolysis of PAH (60), the resultant heterogeneity of which confounds interpretation of the inventors' proposed studies. Working with human PAH cleaved from the fusion protein provided us with an opportunity to assay PAH and to characterize the protein using various chromatographic and electrophoretic methods. FIG. 7A illustrates how human PAH, when purified in this manner, exists as an equilibrium of dimers and tetramers and that the dimer:tetramer ratio is shifted towards the tetramer in the presence of 1 mM Phe, a concentration that the literature suggests is sufficient to fully activate PAH. This shift of the PAH quaternary structure equilibrium could also be shown using native PAGE (not shown). Preliminary studies on the small amount of human protein purified in this fashion showed a specific activity comparable to published values and a dynamic quaternary structure equilibrium that responded to both temperature and ionic strength.

The inventors have also expressed untagged PAH from both human and rat. Although expression was good for both proteins, it was better for rat PAH, which was used for a preliminary purification/characterization using the Shiman affinity method. The purification alone provided some kinetic information on the time required for the high-affinity forms (in the presence of Phe) to relax to the low-affinity forms (in the absence of Phe). Using this method, ˜20 mg of rat PAH (from a 1 liter expression culture) was purified with a specific activity of ˜5 μmol tyrosine mg-1 min-1, which is comparable to published values. For these initial studies, a direct detection fluorescence-based continuous real-time PAH activity assay was used (FIG. 7B) (131). Fortunately, as long as DTT and catalase are present in the assay mixture, there is no need for any special anaerobic environment to maintain this Fe containing-protein in an active state. FIG. 7B illustrates several aspects of PAH kinetics that are consistent with the morpheein model of allosteric regulation (16, 19, 94). Most notable is the dependence of the initial velocity on the order of addition of reaction components. The illustrated experiment, carried out at a Phe concentration sufficient to partially saturate the allosteric site, is consistent with Phe acting to stabilize the active conformations and BH4 acting to stabilize the inactive conformations.

With regard to the oligomeric equilibrium, rat PAH (in 15% glycerol) is found to be predominantly tetrameric by both native PAGE and SEC (FIGS. 7C & 7D). However, native PAGE shows two bands in the tetramer region (FIG. 7C) and the SEC profile hints at the existence of two different tetramers whose ratio responds to Phe (FIG. 7D). Most significantly, FIG. 7E shows the behavior of purified rat PAH on an ion exchange column, where addition of 1 mM Phe to the elution buffer causes a dramatic shift in the elution profile. Coupled with the SEC results in FIG. 7D, this illustrates that IEC can be used to separate two tetramers, 4mer and 4mer*. Most notably, using 50 μM Phe in the elution buffer shows a mixture of both forms as well as a slower eluting form that was not present in the earlier samples. FIG. 7E shows that incubation of ˜1 mg/ml rat PAH with Phe and/or BH4 alters the distribution of PAH quaternary structure isoforms as monitored by native PAGE.

Purified human PAH has a higher propensity to exist in the dimeric forms relative to the rat PAH (compare FIGS. 7A and 7D). A combination of the IEC method and native PAGE can be optimized to quantify the ratio of human PAH quaternary structure isoforms for both wild type and disease-associated variants, as described below.

### Example 4

### Relationship Between PAH Quaternary Structure Equilibrium and Variants Associated with Hyperphenylalaninemia and PKU

PKU-associated PAH variants as defective in the modulation of an equilibrium of alternate multimers is complementary to an alternate view of PKU as a conformational disease where the defect lies in protein folding and subsequent oligomerization (e.g. (6, 7, 61)). In the presented models, the individual PAH domains are not necessarily defective in their ability to properly fold. The defect is in factors that affect the thermodynamics and kinetics of the transitions (e.g. [symbol] ) between functionally distinct multimers (e.g. 4mer* [symbol] 2mer* [symbol] 2mer [symbol] 4mer). The subunits in the off-states (2mer* and 4mer*) are not misfolded; they are in a physiologically relevant inactive conformation. The subunits in “aggregates” observed for some disease associated variants are proposed to exist as nmer* as illustrated for a 6mer* in FIG. 6.

Disease-associated human PAH variants is created using the QuikChange method (14, 11-13, 123, 132-134). Variant proteins are expressed without tags and analyzed using methods discussed above. Proportions of the various oligomers are determined by chromatographic separation of isoforms during purification. FIG. 7E, which shows using IEC to monitor the sensitivity of the PAH quaternary structure equilibrium to various concentrations of Phe, is a straightforward way to compare variants by monitoring the sensitivity of their equilibria to perturbation by a fixed sub-saturating concentration of Phe. The kinetic, biochemical, and biophysical properties of the resulting separated fractions are correlated with alternate multimers as described above. Focus will be on common disease-associated missense mutations (R408W, I65T, R261Q, P281L, R158Q, E280K, Y414C, R252W, L48S), mutations at a domain-domain interface of the 4mer* model (F39L, S110L, R243Q, L249F, A313T, Y377C, T378S), at inter-domain hinge regions (F410S, R413P), and at rare mutations that respond to BH4 (R68S, H170D, E 178G, V190A, A300S, L308F, A373T, E390G, and P4075) (4, 30, 31-32, 76, 120).

Ex post facto inspection of the preliminary 4mer model gratifyingly revealed that the PKU-associated variants G46S, A47T, T63P/H64N, I65T, and R68S, which have been reported to eliminate Phe binding to a PAH fusion protein (57), all exist near the proposed ACT-ACT interface of 4mer. Because the inventors target the entire ACT-ACT interface for discovery/development of pharmacologic chaperones, it is possible that these variants respond to allosteric pharmacological chaperones even though they do not respond to allosteric Phe binding. The methods and procedures are straightforward. The inventors provide the preparation and characterization of most, if not all, of the above described variants, and the determination of how the position of the quaternary structure equilibrium responds to ligands such as Phe, BH4, and their analogs.

### Example 5

### Various Models for the Allosteric Regulation of PAH

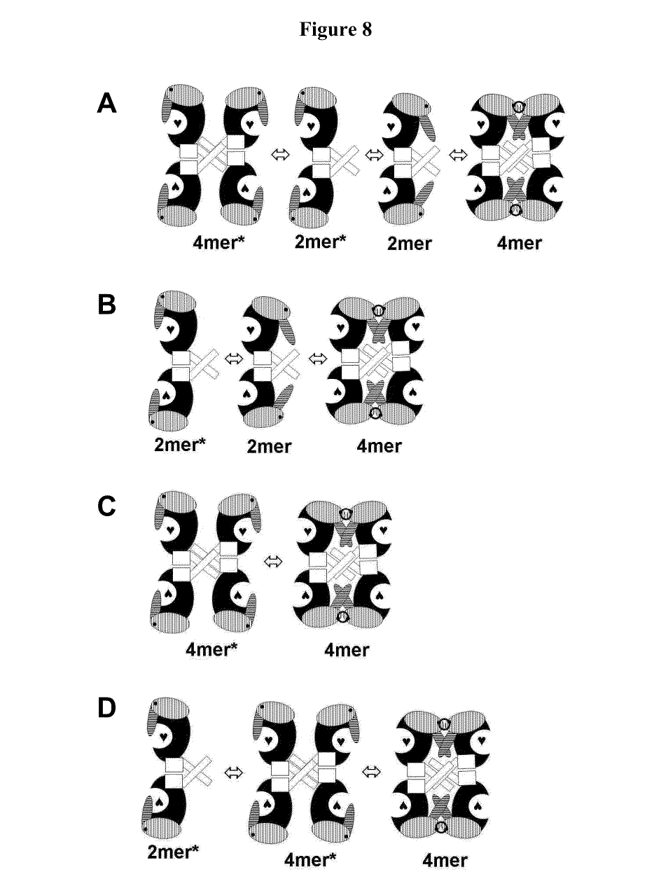

Because PAH active sites do not exist at a subunit-subunit interface, the simple 2 mer (low-activity) [symbol] 4mer (high-activity) model is discounted. Given the evidence for at least one kind of tetramer and for an equilibrium of dimers and tetramers, FIG. 8 illustrates several viable models for PAH allostery. These include two morpheein models (4mer* [symbol] 2mer* [symbol] 2mer [symbol] 4mer and 2mer* [symbol] 2mer [symbol] 4mer), where the essential “*” conformational change must occur in the dissociated state, one classic Monod-Wyman-Changeux concerted model (4mer* [symbol] 4mer) that does not require participation of a dimeric assembly, and one dissociative model (2mer* [symbol] 4mer* [symbol] 4mer) where the low-activity dimer is not on the pathway between a low-activity and high-activity tetramer. Key to discriminating among these models is to determine whether there are two different kinds of tetramers, which is strongly supported by the data the inventors present in FIGS. 7C, 7D, 7E, & 7F and to address whether the interchange between these tetramers requires a dissociation step.

Using the methods detailed above, the invention provides the separation of the isoforms putatively identified as 4mer and 4mer* and the confirmation that their conformational identity with the well established fluorescence and limited proteolysis methods. A subunit disproportionation experiment as the inventors have done before (12, 19) determines whether tetramer dissociation to dimer is an essential part of allostery. The inventors' work with mixed oligomers of PBGS provides the precedent for establishing or discounting the dissociative nature of the PAH transitions. Using a bicistronic tandem expression system, the inventors prepared mixed oligomers of wild type PBGS (predominantly 8mer) and the disease-associated F12L variant (predominantly 6mer*) (11, 12). The hetero-8mers and hetero-6mer*s, each containing chains with both Phe12 and Leu12, were separated by ion exchange chromatography and analyzed by mass spectroscopy to determine the mole fraction of Phe12 and Leu12 in each. As illustrated in FIG. 9, the isolated hetero-6mer* was then dialyzed against assay buffer containing substrate, which favors formation of 8mer. After partial conversion of hetero-6mer* to hetero-8mer, the protein was removed from the dialysis chamber and the resulting hetero-8mer and hetero-6mer* were again separated on an ion exchange column. The resulting pools were reanalyzed for the mole fractions of Phe12- and Leu12-containing chains. The produced 8mer was enriched in Phe12, while the remaining 6mer* was enriched in Leu12, thus showing that the individual chains had disproportionated and unequivocally establishing the dissociative nature of the interchange between 6mer* and 8mer (12).

The key to repeating this experiment for PAH is to identify (or design) a PAH variant that has a high propensity to exist as 4mer* under conditions where the wild type protein exists mostly as 4mer. Fortunately, it is established that bicistronic tandem expression of variant PAH proteins results in hetero-oligomers (83). Many human PAH variants identified in the invention have been described in the literature which might favor 4mer*. Some mutations are at positions that lie at hinge regions of the structure, which are the likely locations of a backbone twist in the interdomain reorganization 2mer [symbol] 2mer* transition (or 4mer [symbol] 4mer*, if this is not forbidden by steric hindrance). For example, T427P has been shown to dramatically shift the dimer-tetramer equilibrium and does not show kinetic cooperativity typical of PAH (4). The inventors' designed variants T117P and G33P are candidates predicted to limit rotation at hinges in the regulatory domain by mutation to proline. Prior design success is followed wherein removing an arginine at a dissociable hydrated/hydrophilic interface dramatically shifted the PBGS 8mer/6mer* equilibrium. In PAH, Arg261 (where R261Q is disease-associated) and Arg243 (where R243Q is disease-associated) are at the interface between the catalytic domain and the regulatory domains. Although X-ray crystallography is not a specific aim of the invention, the crystal structure of the PAH variants that strongly favor one quaternary isoform may be instructive.

### Example 6

### Small Molecules Modulators of PAH Quaternary Structure Equilibrium (Equilibrium of Morpheein Forms) and Activity

Small-molecule perturbation of quaternary structure equilibria is considered as a general mechanism for drug action and side effects; here the inventors test this hypothesis with human PAH. Although the long term goal of the inventors' work with PAH is to identify pharmacological chaperones that act to stabilize the active conformations 4mer (and perhaps also 2mer), the inventors may also identify compounds that stabilize the inactive conformations. These drugs could be contraindicated in PKU and hyperphenylalaninemia patients.

The inventors provide a native PAGE mobility shift screen of the JHCCL and NTP libraries against human PAH, following procedures established for PBGS (18, 142) by the inventors. Because human PAH exists as an equilibrium containing substantial amounts of both tetramer and dimer, the initial screen looks for compounds that can perturb this ratio (as demonstrate for Phe in FIG. 7A). Human PAH has a higher propensity to exist as a dimer, relative to the rat protein. This propensity contributes to its reputation as the “more difficult” or “less stable” of the mammalian PAHs. Although it is straightforward to monitor the dimer:tetramer ratio, it may also prove possible to monitor the ratio of the two closely spaced tetrameric bands that are two architecturally different tetramers (4mer and 4mer*) (FIG. 7C).

The quaternary structure modulators that favor a specific oligomer are tested to determine whether these compounds can modulate the activity of PAH (activation and/or inhibition), and to quantify the potency of these putative allosteric effectors as the inventors have done before (18). Together the two cited libraries contain ˜2900 compounds. A compound collection of such a size can be manually screened, in duplicate using the PhastGel technology (two instruments). The invention is guided by the knowledge that the PAH quaternary structure equilibrium is sufficiently fragile to respond to changes pH, temperature, and ionic strength. Small molecules structurally unrelated to Phe that can modulate the human PAH quaternary structure equilibrium are identified with one caveat that it must be insured that small molecule stabilization of the ACT-ACT interface is selective for 4mer stabilization and does not promote aggregation as illustrated in FIG. 6.

### REFERENCES

1. Blau N, van Spronsen F J, Levy H L. Phenylketonuria. Lancet. 2010; 376(9750):1417-27.
2. Bjorgo E, de Carvalho R M, Flatmark T. A comparison of kinetic and regulatory properties of the tetrameric and dimeric forms of wild-type and Thr427->Pro mutant human phenylalanine hydroxylase: contribution of the flexible hinge region Asp425-Gln429 to the tetramerization and cooperative substrate binding. Eur J. Biochem. 2001; 268(4):997-1005.
3. Gamez A, Perez B, Ugarte M, Desviat L R. Expression analysis of phenylketonuria mutations. Effect on folding and stability of the phenylalanine hydroxylase protein. J Biol. Chem. 2000; 275(38):29737-42.
4. Stokka A J, Carvalho R N, Barroso J F, Flatmark T. Probing the role of crystallographically defined/predicted hinge-bending regions in the substrate-induced global conformational transition and catalytic activation of human phenylalanine hydroxylase by single-site mutagenesis. J Biol. Chem. 2004; 279(25):26571-80.
5. Jaffe E K. Morpheeins—a new structural paradigm for allosteric regulation. Trends in Biochemical Sciences. 2005; 30(9):490-7.
6. Leandro J, Simonsen N, Saraste J, Leandro P, Flatmark T. Phenylketonuria as a protein misfolding disease: The mutation pG46S in phenylalanine hydroxylase promotes self-association and fibril formation. Biochim Biophys Acta. 2011; 1812(1):106-20.
7. Gersting S W, Kemter K F, Staudigl M, Messing D D, Danecka M K, Lagler F B, et al. Loss of function in phenylketonuria is caused by impaired molecular motions and conformational instability. Am J Hum Genet. 2008; 83(1):5-17. PMCID: 2443833.
8. Gersting S W, Lagler F B, Eichinger A, Kemter K F, Danecka M K, Messing D D, et al. Pahenul is a mouse model for tetrahydrobiopterin-responsive phenylalanine hydroxylase deficiency and promotes analysis of the pharmacological chaperone mechanism in vivo. Hum Mol. Genet. 2010; 19(10):2039-49.
9. Jaffe E K. Morpheeins, a new pathway for allosteric drug discovery. The Open Conference Proceedings Journal. 2010; 1:1-6. PMCID: NIHMSID #207695.
10. Watson M S, Mann M Y, Lloyd-Puryear M A, Rinaldo P, Howell R R. Newborn screening: toward a uniform screening panel and system—executive summary. Pediatrics. 2006; 117 (5 Pt 2):5296-307.
11. Breinig S, Kervinen J, Stith L, Wasson A S, Fairman R, Wlodawer A, et al. Control of tetrapyrrole biosynthesis by alternate quaternary forms of porphobilinogen synthase. Nature Structural Biology. 2003; 10(9):757-63.
12. Tang L, Stith L, Jaffe E K. Substrate-induced interconversion of protein quaternary structure isoforms. Journal of Biological Chemistry. 2005; 280(16):15786-93.
13. Tang L, Breinig S, Stith L, Mischel A, Tannir J, Kokona B, et al. Single amino acid mutations alter the distribution of human porphobilinogen synthase quaternary structure isoforms (morpheeins). Journal of Biological Chemistry. 2006; 281(10):6682-90.
14. Jaffe E K, Stith L. ALAD porphyria is a conformational disease. American Journal of Human Genetics. 2007; 80(2):329-37.
15. Kokona B, Rigotti D J, Wasson A S, Lawrence S H, Jaffe E K, Fairman R. Probing the oligomeric assemblies of pea porphobilinogen synthase by analytical ultracentrifugation. Biochemistry. 2008; 47(40):10649-56. PMCID: 2559947.
16. Lawrence S H, Jaffe E K. Expanding the Concepts in Protein Structure-Function Relationships and Enzyme Kinetics: Teaching using Morpheeins. Biochem Mol Biol Educ. 2008; 36(4):274-83. PMCID: 2575429.
17. Selwood T, Tang L, Lawrence S H, Anokhina Y, Jaffe E K. Kinetics and thermodynamics of the interchange of the morpheein forms of human porphobilinogen synthase. Biochemistry. 2008; 47(10):3245-57.
18. Lawrence S H, Selwood T, Jaffe E K. Diverse Clinical Compounds Alter the Quaternary Structure and Inhibit the Activity of an Essential Enzyme. ChemMedChem. 2011; (epub ahead of print 2011/04/21). PMCID: in process.
19. Jaffe E K, Lawrence S H. The morpheein model of allostery: Evaluating proteins as potential morpheeins. In: Fenton A, editor. Methods in Molecular Biology—Monitoring Allosteric Function: Springer; 2011. p. (in press).
20. Puy H, Gouya L, Deybach J C. Porphyrias. Lancet. 2010; 375(9718):924-37.
21. Fitzpatrick P F. The aromatic amino acid hydroxylases. Adv Enzymol Relat Areas Mol. Biol. 2000; 74:235-94.
22. Ayling J, Pirson R, Pirson W, Boehm G. A specific kinetic assay for phenylalanine hydroxylase. Anal Biochem. 1973; 51(1): 80-90.
23. Woo S L, Gillam S S, Woolf L I. The isolation and properties of phenylalanine hydroxylase from human liver. Biochem J. 1974; 139(3):741-9. PMCID: 1166338.
24. Shiman R, Gray D W. Substrate activation of phenylalanine hydroxylase. A kinetic characterization. J Biol. Chem. 1980; 255(10):4793-800.
25. Ledley F D, Grenett H E, Woo S L. Biochemical characterization of recombinant human phenylalanine hydroxylase produced in Escherichia coli. J Biol. Chem. 1987; 262(5):2228-33.
26. Shiman R, Jones S H, Gray D W. Mechanism of phenylalanine regulation of phenylalanine hydroxylase. J Biol. Chem. 1990; 265(20):11633-42.
27. Knappskog P M, Haavik J. Tryptophan fluorescence of human phenylalanine hydroxylase produced in Escherichia coli. Biochemistry. 1995; 34(37):11790-9.
28. Kowlessur D, Citron B A, Kaufman S. Recombinant human phenylalanine hydroxylase: novel regulatory and structural properties. Arch Biochem Biophys. 1996; 333(1):85-95.
29. Kaufman S. The phenylalanine hydroxylating system. Adv Enzymol Relat Areas Mol. Biol. 1993; 67:77-264.
30. Fusetti F, Erlandsen H, Flatmark T, Stevens R C. Structure of tetrameric human phenylalanine hydroxylase and its implications for phenylketonuria. J Biol. Chem. 1998; 273(27):16962-7.
31. Scriver C R, Waters P J, Sarkissian C, Ryan S, Prevost L, Cote D, et al. PAHdb: a locus-specific knowledgebase. Hum Mutat. 2000; 15(1):99-104.
32. Scriver C R, Hurtubise M, Konecki D, Phommarinh M, Prevost L, Erlandsen H, et al. PAHdb 2003: what a locus-specific knowledgebase can do. Hum Mutat. 2003; 21(4):333-44.
33. Lawrence S H, Ramirez U D, Tang L, Fazliyez F, Kundrat L, Markham G D, et al. Shape shifting leads to small-molecule allosteric drug discovery. Chem. Biol. 2008; 15(6):586-96. PMCID: 2703447.
34. Lawrence S H, Ramirez U D, Selwood T, Stith L, Jaffe E K. Allosteric inhibition of human porphobilinogen synthase. J Biol. Chem. 2009; 284(51):35807-17. PMCID: 2791010.
35. Ramirez U D, Myachina F, Stith L, Jaffe E K. Docking to Large Allosteric Binding Sites on Protein Surfaces. Adv Exp Med. Biol. 2010; 680:481-8. PMCID: 2946403.
36. Reitz A B, Ramirez U D, Stith L, Du Y, Smith G R, Jaffe E K. Pseudomonas aeruginosa porphobilinogen synthase Assembly State Regulators: hit discovery and initial SAR studies. ARKIVOC. 2010; 2010 (viii):175-88. PMCID: NIHMS218463.
37. Lawrence S H, Selwood T, Jaffe E K. Diverse clinical compounds alter the quaternary structure and inhibit the activity of an essential enzyme. ChemMedChem. 2011:April 19, E-pub before print. PMCID: being processed.
38. van Spronsen F J. Phenylketonuria: a 21st century perspective. Nat Rev Endocrinol. 2010; 6(9):509-14.
39. van Spronsen F J, Enns G M. Future treatment strategies in phenylketonuria. Mol Genet Metab. 2010; 99 Suppl 1:S90-5.
40. Harding C O, Gillingham M B, Hamman K, Clark H, Goebel-Daghighi E, Bird A, et al.

Complete correction of hyperphenylalaninemia following liver-directed, recombinant AAV2/8 vector-mediated gene therapy in murine phenylketonuria. Gene Ther. 2006; 13(5):457-62. PMCID: 2813194.
41. Eisensmith R C, Woo S L. Gene therapy for phenylketonuria. Eur J. Pediatr. 1996; 155 Suppl 1:S16-9.
42. Kang T S, Wang L, Sarkissian C N, Gamez A, Scriver C R, Stevens R C. Converting an injectable protein therapeutic into an oral form: phenylalanine ammonia lyase for phenylketonuria. Mol Genet Metab. 2010; 99(1):4-9. PMCID: 2795033.
43. Sarkissian C N, Shao Z, Blain F, Peevers R, Su H, Heft R, et al. A different approach to treatment of phenylketonuria: phenylalanine degradation with recombinant phenylalanine ammonia lyase. Proc Natl Acad Sci USA. 1999; 96(5):2339-44. PMCID: 26785.
44. Bourget L, Chang T M. Phenylalanine ammonia-lyase immobilized in semipermeable microcapsules for enzyme replacement in phenylketonuria. Febs Lett. 1985; 180(1):5-8.
45. Hoskins J A, Gray J. Phenylalanine ammonia lyase in the management of phenylketonuria: the relationship between ingested cinnamate and urinary hippurate in humans. Res Commun Chem Pathol Pharmacol. 1982; 35(2):275-82.
46. Andersen A E, Avins L. Lowering brain phenylalanine levels by giving other large neutral amino acids. A new experimental therapeutic approach to phenylketonuria. Arch Neurol. 1976; 33(10):684-6.
47. Pietz J, Kreis R, Rupp A, Mayatepek E, Rating D, Boesch C, et al. Large neutral amino acids block phenylalanine transport into brain tissue in patients with phenylketonuria. J Clin Invest. 1999; 103(8):1169-78. PMCID: 408272.
48. Moats R A, Moseley K D, Koch R, Nelson M, Jr. Brain phenylalanine concentrations in phenylketonuria: research and treatment of adults. Pediatrics. 2003; 112 (6 Pt 2):1575-9.
49. Schindeler S, Ghosh-Jerath S, Thompson S, Rocca A, Joy P, Kemp A, et al. The effects of large neutral amino acid supplements in PKU: an MRS and neuropsychological study. Mol Genet Metab. 2007; 91(1):48-54.
50. Knappskog P M, Flatmark T, Aarden J M, Haavik J, Martinez A. Structure/function relationships in human phenylalanine hydroxylase. Effect of terminal deletions on the oligomerization, activation and cooperativity of substrate binding to the enzyme. Eur J. Biochem. 1996; 242(3):813-21.
51. Leandro J, Nascimento C, de Almeida I T, Leandro P. Co-expression of different subunits of human phenylalanine hydroxylase: evidence of negative interallelic complementation. Biochim Biophys Acta. 2006; 1762(5):544-50.
52. Jennings I G, Cotton R G, Kobe B. Structural interpretation of mutations in phenylalanine hydroxylase protein aids in identifying genotype-phenotype correlations in phenylketonuria. Eur J Hum Genet. 2000; 8(9):683-96.
53. Waters P J. How PAH gene mutations cause hyper-phenylalaninemia and why mechanism matters: insights from in vitro expression. Hum Mutat. 2003; 21(4):357-69.
54. Waters P J, Scriver C R, Parniak M A. Homomeric and heteromeric interactions between wild-type and mutant phenylalanine hydroxylase subunits: evaluation of two-hybrid approaches for functional analysis of mutations causing hyperphenylalaninemia. Mol Genet Metab. 2001; 73(3):230-8.
55. Waters P J, Parniak M A, Nowacki P, Scriver C R. In vitro expression analysis of mutations in phenylalanine hydroxylase: linking genotype to phenotype and structure to function. Hum Mutat. 1998; 11(1):4-17.
56. Desviat L R, Perez B, Garcia M J, Martinez-Pardo M, Baldellou A, Arena J, et al. Relationship between mutation genotype and biochemical phenotype in a heterogeneous Spanish phenylketonuria population. Eur J Hum Genet. 1997; 5(4):196-202.
57. Gjetting T, Petersen M, Guldberg P, Guttler F. In vitro expression of 34 naturally occurring mutant variants of phenylalanine hydroxylase: correlation with metabolic phenotypes and susceptibility toward protein aggregation. Mol Genet Metab. 2001; 72(2):132-43.
58. Jaffe E K. The porphobilinogen synthase catalyzed reaction mechanism. Bioorganic Chemistry. 2004; 32(5):316-25.
59. Phillips R S, Parniak M A, Kaufman S. Spectroscopic investigation of ligand interaction with hepatic phenylalanine hydroxylase: evidence for a conformational change associated with activation. Biochemistry. 1984; 23(17):3836-42.
60. Martinez A, Knappskog P M, Olafsdottir S, Doskeland A P, Eiken H G, Svebak R M, et al. Expression of recombinant human phenylalanine hydroxylase as fusion protein in Escherichia coli circumvents proteolytic degradation by host cell proteases. Isolation and characterization of the wild-type enzyme. Biochem J. 1995; 306 (Pt 2):589-97. PMCID: 1136558.
61. Bjorgo E, Knappskog P M, Martinez A, Stevens R C, Flatmark T. Partial characterization and three-dimensional-structural localization of eight mutations in exon 7 of the human phenylalanine hydroxylase gene associated with phenylketonuria. Eur J. Biochem. 1998; 257(1):1-10.
62. Waters P J, Parniak M A, Hewson A S, Scriver C R. Alterations in protein aggregation and degradation due to mild and severe missense mutations (A104D, R157N) in the human phenylalanine hydroxylase gene (PAH). Hum Mutat. 1998; 12(5):344-54.
63. Kobe B, Jennings I G, House C M, Michell B J, Goodwill K E, Santarsiero B D, et al. Structural basis of autoregulation of phenylalanine hydroxylase. Nat Struct Biol. 1999; 6(5):442-8.
64. Frieden C. Kinetic Aspects of Regulation of Metabolic Processes—Hysteretic Enzyme Concept. Journal of Biological Chemistry. 1970; 245(21):5788-5799.
65. Jaffe E K. An unusual phylogenetic variation in the metal ion binding sites of porphobilinogen synthase. Chemistry & Biology. 2003; 10(1):25-34.
66. Li J, Ilangovan U, Daubner S C, Hinck A P, Fitzpatrick P F. Direct evidence for a phenylalanine site in the regulatory domain of phenylalanine hydroxylase. Arch Biochem Biophys. 2011; 505(2):250-5. PMCID: 3019263.
67. Zurfluh M R, Zschocke J, Lindner M, Feillet F, Chery C, Burlina A, et al. Molecular genetics of tetrahydrobiopterin-responsive phenylalanine hydroxylase deficiency. Hum Mutat. 2008; 29(1):167-75.
68. Shiman R, Gray D W, Pater A. A simple purification of phenylalanine hydroxylase by substrate-induced hydrophobic chromatography. J Biol. Chem. 1979; 254(22):11300-6.
69. Jaffe E K, Shanmugam D, Gardberg A, Dieterich S, Sankaran B, Stewart L J, et al. Crystal Structure of Toxoplasma gondii Porphobilinogen Synthase: INSIGHTS ON OCTAMERIC STRUCTURE AND PORPHOBILINOGEN FORMATION. J Biol. Chem. 2011; 286(17):15298-307. PMCID: 3083160.
70. Fitzpatrick P F. Mechanism of aromatic amino acid hydroxylation. Biochemistry. 2003; 42(48):14083-91. PMCID: 1635487.
71. Pavon J A, Fitzpatrick P F. Insights into the catalytic mechanisms of phenylalanine and tryptophan hydroxylase from kinetic isotope effects on aromatic hydroxylation. Biochemistry. 2006; 45(36):11030-7. PMCID: 1945167.
72. Andersen O A, Flatmark T, Hough E. High resolution crystal structures of the catalytic domain of human phenylalanine hydroxylase in its catalytically active Fe(II) form and binary complex with tetrahydrobiopterin. J Mol. Biol. 2001; 314(2):279-91.
73. Andersen O A, Flatmark T, Hough E. Crystal structure of the ternary complex of the catalytic domain of human phenylalanine hydroxylase with tetrahydrobiopterin and 3-(2-thienyl)-L-alanine, and its implications for the mechanism of catalysis and substrate activation. J Mol. Biol. 2002; 320(5):1095-108.
74. Andersen O A, Stokka A J, Flatmark T, Hough E. 2.0A resolution crystal structures of the ternary complexes of human phenylalanine hydroxylase catalytic domain with tetrahydrobiopterin and 3-(2-thienyl)-L-alanine or L-norleucine: substrate specificity and molecular motions related to substrate binding. J Mol. Biol. 2003; 333(4):747-57.
75. Erlandsen H, Flatmark T, Stevens R C, Hough E. Crystallographic analysis of the human phenylalanine hydroxylase catalytic domain with bound catechol inhibitors at 2.0 A resolution. Biochemistry. 1998; 37(45):15638-46.
76. Erlandsen H, Pey A L, Gamez A, Perez B, Desviat L R, Aguado C, et al. Correction of kinetic and stability defects by tetrahydrobiopterin in phenylketonuria patients with certain phenylalanine hydroxylase mutations. Proc Natl Acad Sci USA. 2004; 101(48):16903-8. PMCID: 534739.
77. Liberles J S, Thorolfsson M, Martinez A. Allosteric mechanisms in ACT domain containing enzymes involved in amino acid metabolism. Amino Acids. 2005; 28(1):1-12.
78. Ting D, Wang G, Shapovalov M, Mitra R, Jordan M I, Dunbrack R L, Jr. Neighbor-dependent Ramachandran probability distributions of amino acids developed from a hierarchical Dirichlet process model. PLoS Comput Biol. 2010; 6 (4):e1000763. PMCID: 2861699.
79. Krivov G G, Shapovalov M V, Dunbrack R L, Jr. Improved prediction of protein side-chain conformations with SCWRL4. Proteins. 2009; 77(4):778-95. PMCID: 2885146.
80. Wang Q, Canutescu A A, Dunbrack R L, Jr. SCWRL and Moll D E: computer programs for side-chain conformation prediction and homology modeling. Nat. Protoc. 2008; 3(12):1832-47. PMCID: 2682191.
81. Dunbrack R L, Jr. Sequence comparison and protein structure prediction. Curr Opin Struct Biol. 2006; 16(3):374-84.
82. Canutescu A A, Dunbrack R L, Jr. Moll D E: a homology modeling framework you can click with. Bioinformatics. 2005; 21(12):2914-6.
83. Leandro J, Leandro P, Flatmark T. Heterotetrameric forms of human phenylalanine hydroxylase: Co-expression of wild-type and mutant forms in a bicistronic system. Biochim Biophys Acta. 2011; 1812(5):602-12.
84. Papenbrock, J. et al. Role of magnesium chelatase activity in the early steps of the tetrapyrrole biosynthetic pathway. Plant Physiol (2000) 122 (4), 1161-1169
85. Martinez A, Calvo A C, Teigen K, Pey A L. Rescuing proteins of low kinetic stability by chaperones and natural ligands phenylketonuria, a case study. Prog Mol Biol Transl Sci. 2008; 83:89-134.
86. Nascimento C, Leandro J, Tavares de Almeida I, Leandro P. Modulation of the activity of newly synthesized human phenylalanine hydroxylase mutant proteins by low-molecular-weight compounds. Protein J. 2008; 27(6):392-400.
87. Pey A L, Ying M, Cremades N, Velazquez-Campoy A, Scherer T, Thony B, Sancho J, Martinez A. Identification of pharmacological chaperones as potential therapeutic agents to treat phenylketonuria. J Clin Invest. 2008; 118(8):2858-67. PMCID: 2441854.
88. Muntau A C, Gersting S W. Phenylketonuria as a model for protein misfolding diseases and for the development of next generation orphan drugs for patients with inborn errors of metabolism. J Inherit Metab Dis. 2010; 33(6):649-58.
89. Leandro J, Saraste J, Leandro P, Flatmark T. The G46S-hPAH mutant protein: a model to study the rescue of aggregation-prone PKU mutations by chaperones. Mol Genet Metab. 2011; 104 Suppl:S40-4.
90. Staudigl M, Gersting S W, Danecka M K, Messing D D, Woidy M, Pinkas D, Kemter K F, Blau N, Muntau A C. The interplay between genotype, metabolic state and cofactor treatment governs phenylalanine hydroxylase function and drug response. Hum Mol. Genet. 2011; 20(13):2628-41.
91. Santos-Sierra S, Kirchmair J, Perna A M, Reiss D, Kemter K, Roschinger W, Glossmann H, Gersting S W, Muntau A C, Wolber G, Lagler F B. Novel pharmacological chaperones that correct phenylketonuria in mice. Hum Mol. Genet. 2012; 21(8):1877-87.
92. Torreblanca R, Lira-Navarrete E, Sancho J, Hurtado-Guerrero R. Structural and Mechanistic Basis of the Interaction between a Pharmacological Chaperone and Human Phenylalahine Hydroxylase. Chembiochem. 2012.
93. Ringe D, Petsko G A. What are pharmacological chaperones and why are they interesting? J Biol. 2009; 8(9):80. PMCID: 2776907.
94. Selwood T, Jaffe E K. Dynamic dissociating homo-oligomers and the control of protein function. Archives of Biochemistry and Biophysics. 2012; 519(2):131-43. PMCID: 3298769.
95. Scriver C R, Clow C L. Phenylketonuria: epitome of human biochemical genetics (first of two parts). N Engl J. Med. 1980; 303(23):1336-42
96. Posner J, Gorman D, Nagel B J. Tyrosine supplements for ADHD symptoms with comorbid phenylketonuria. J Neuropsychiatry Clin Neurosci. 2009; 21(2):228-30.
97. Antshel K M. ADHD, learning, and academic performance in phenylketonuria. Mol Genet Metab. 2010; 99 Suppl 1:S52-8.
98. Jaffe E K, Lawrence S H. Allostery and the dynamic oligomerization of porphobilinogen synthase. Archives of Biochemistry and Biophysics. 2012; 519(2):144-53. PMCID: 3291741.
99. Chuang D T. Maple syrup urine disease: it has come a long way. J. Pediatr. 1998; 132 (3 Pt 2):517-23.
100. Chuang D T, Shih V E. Maple syrup urine disease (branched-chain ketoaciduria). In: Scriver C R, Beaudet A L, Sly W S, Valle D, editors. The Metabolic and Molecular Basis of Inherited Disease. 8th Ed. ed. New York: McGraw-Hill; 2001. p. 1971-2005.
101. Odievre M H, Chretien D, Munnich A, Robinson B H, Dumoulin R, Masmoudi S, Kadhom N, Rotig A, Rustin P, Bonnefont J P. A novel mutation in the dihydrolipoamide dehydrogenase E3 subunit gene (DLD) resulting in an atypical form of alpha-ketoglutarate dehydrogenase deficiency. Hum Mutat. 2005; 25(3):323-4.
102. Miles E W, Kraus J P. Cystathionine beta-synthase: structure, function, regulation, and location of homocystinuria-causing mutations. J Biol. Chem. 2004; 279(29):29871-4.
103. Mudd S H. Hypermethioninemias of genetic and non-genetic origin: A review. Am J Med Genet C Semin Med. Genet. 2011; 157(1):3-32.
104. Trompier D, Alibert M, Davanture S, Hamon Y, Pierres M, Chimini G. Transition from dimers to higher oligomeric forms occurs during the ATPase cycle of the ABCA1 transporter. J Biol. Chem. 2006; 281(29):20283-90.
105. Gao X, Wang H, Yang J J, Liu X, Liu Z R. Pyruvate kinase m2 regulates gene transcription by acting as a protein kinase. Mol. Cell. 2012; 45(5):598-609. PMCID: 3299833.
106. Semenova G, ChemoffJ. PKM2 Enters the Morpheein Academy. Mol. Cell. 2012; 45(5):583-4.
107. Yang Z, Lanks C W, Tong L. Molecular mechanism for the regulation of human mitochondrial NAD(P)+—dependent malic enzyme by ATP and fumarate. Structure. 2002; 10(7):951-60.
108. Palenchar J B, Colman R F. Characterization of a mutant Bacillus subtilis adenylosuccinate lyase equivalent to a mutant enzyme found in human adenylosuccinate lyase deficiency: asparagine 276 plays an important structural role. Biochemistry. 2003; 42(7):1831-41.
109. Scriver C R, Katz L, Clow C. Phenylketonuria and diet. Can Med Assoc J. 1968; 98(2):124-5. PMCID: 1923756.
110. Scriver C R, Clow C L. Phenylketonuria: epitome of human biochemical genetics (second of two parts). N Engl J. Med. 1980; 303(24):1394-400.
111. Azadi B, Seddigh A, Tehrani-Doost M, Alaghband-Rad J, Ashrafi M R. Executive dysfunction in treated phenylketonuric patients. Eur Child Adolesc Psychiatry. 2009; 18(6):360-8.
112. Woolf L I, Griffiths R, Moncrieff A. Treatment of phenylketonuria with a diet low in phenylalanine. Br Med J. 1955; 1(4905):57-64. PMCID: 2060789.
113. Horner F A, Streamer C W. Effect of a phenylalanine-restricted diet on patients with phenylketonuria; clinical observations in three cases. J Am Med. Assoc. 1956; 161(17):1628-30.
114. Horner F A, Streamer C W, Clader D E, Hassell L L, Binkley E L, Jr., Dumars K W, Jr. Effect of phenylalanine-restricted diet in phenylketonuria. II. AMA J Dis Child. 1957; 93(6):615-8.
115. Hsia D Y, Knox W E, Quinn K V, Paine R S. A one-year, controlled study of the effect of low-phenylalanine diet on phenylketonuria. Pediatrics. 1958; 21(2): 178-202.
116. van Spronsen F J, de Groot M J, Hoeksma M, Reijngoud D J, van Rijn M. Large neutral amino acids in the treatment of PKU: from theory to practice. J Inherit Metab Dis. 2010; 33(6):671-6. PMCID: 2992655.
117. Pey A L, Perez B, Desviat L R, Martinez M A, Aguado C, Erlandsen H, Gamez A, Stevens R C, Thorolfsson M, Ugarte M, Martinez A. Mechanisms underlying responsiveness to tetrahydrobiopterin in mild phenylketonuria mutations. Hum Mutat. 2004; 24(5):388-99.
118. Miller S R, Sekijima Y, Kelly J W. Native state stabilization by NSAIDs inhibits transthyretin amyloidogenesis from the most common familial disease variants. Lab Invest. 2004; 84(5):545-52
119. Maruno M, Furuyama K, Akagi R, Horie Y, Meguro K, Garbaczewski L, Chiorazzi N, Doss M O, Hassoun A, Mercelis R, Verstraeten L, Harper P, Floderus Y, Thunell S, Sassa S. Highly heterogeneous nature of delta-aminolevulinate dehydratase (ALAD) deficiencies in ALAD porphyria. Blood. 2001; 97(10):2972-8.
120. Matalon R, Koch R, Michals-Matalon K, Moseley K, Surendran S, Tyring S, Erlandsen H, Gamez A, Stevens R C, Romstad A, Moller L B, Guttler F. Biopterin responsive phenylalanine hydroxylase deficiency. Genet Med. 2004; 6(1):27-32.
121. Xu Q, Dunbrack R L, Jr. The protein common interface database (ProtCID)—a comprehensive database of interactions of homologous proteins in multiple crystal forms. Nucleic Acids Res. 2011; 39 (Database issue):D761-70. PMCID: 3013667.
122. Kotaka M, Ren J, Lockyer M, Hawkins A R, Stammers D K. Structures of R- and T-state Escherichia coli aspartokinase III. Mechanisms of the allosteric transition and inhibition by lysine. J Biol. Chem. 2006; 281(42):31544-52.
123. Jaffe E K, Volin M, Bronson-Mullins C R, Dunbrack R L, Kervinen J, Martins J, Quinlan J F, Sazinsky M H, Steinhouse E M, Yeung A T. An artificial gene for human porphobilinogen synthase allows comparison of an allelic variation implicated in susceptibility to lead poisoning. Journal of Biological Chemistry. 2000; 275(4):2619-26.
124. Pohnert G, Zhang S, Husain A, Wilson D B, Ganem B. Regulation of phenylalanine biosynthesis. Studies on the mechanism of phenylalanine binding and feedback inhibition in the Escherichia coli P-protein. Biochemistry. 1999; 38(38):12212-7.
125. Baldwin G S, McKenzie G H, Davidson B E. The self-association of chorismate mutase/prephenate dehydratase from Escherichia coli K12. Arch Biochem Biophys. 1981; 211 (1):76-85.
126. Zhang S, Pohnert G, Kongsaeree P, Wilson D B, Clardy J, Ganem B. Chorismate mutase-prephenate dehydratase from Escherichia coli. Study of catalytic and regulatory domains using genetically engineered proteins. J Biol. Chem. 1998; 273(11):6248-53.
127. Li J, Dangott L J, Fitzpatrick P F. Regulation of phenylalanine hydroxylase: conformational changes upon phenylalanine binding detected by hydrogen/deuterium exchange and mass spectrometry. Biochemistry. 2010; 49(15):3327-35. PMCID: 2855537.
128. Shiman R, Xia T, Hill M A, Gray D W. Regulation of rat liver phenylalanine hydroxylase. II. Substrate binding and the role of activation in the control of enzymatic activity. J Biol. Chem. 1994; 269(40):2464756.
129. Krieger E, Koraimann G, Vriend G. Increasing the precision of comparative models with YASARA NOVA—a self-parameterizing force field. Proteins. 2002; 47(3):393-402.
130. Dosztanyi Z, Csizmok V, Tompa P, Simon I. IUPred: web server for the prediction of intrinsically unstructured regions of proteins based on estimated energy content. Bioinformatics. 2005; 21(16):3433-4.
131. Gersting S W, Staudigl M, Truger M S, Messing D D, Danecka M K, Sommerhoff C P, Kemter K F, Muntau A C. Activation of phenylalanine hydroxylase induces positive cooperativity toward the natural cofactor. J Biol. Chem. 2010; 285(40):30686-97. PMCID: 2945563.
132. Kervinen J, Dunbrack R L, Litwin S, Martins J, Scarrow R C, Volin M, Yeung A T, Yoon E, Jaffe E K. Porphobilinogen synthase from pea: Expression from an artificial gene, kinetic characterization, and novel implications for subunit interactions. Biochemistry. 2000; 39(30):9018-29.
133. Jaffe E K, Martins J, Li J, Kervinen J, Dunbrack R L. The molecular mechanism of lead inhibition of human porphobilinogen synthase. Journal of Biological Chemistry. 2001; 276 (2)1 531-7.
134. Mitchell L W, Volin M, Martins J, Jaffe E K. Mechanistic implications of mutations to the active site lysine of porphobilinogen syntheses. Journal of Biological Chemistry. 2001; 276(2): 1538-44.
135. Carrell H L, Glusker J P, Shimoni L, Keefe L J, Afshar C, Volin M, Jaffe E K. Crystallization and preliminary X-ray diffraction studies of 5-chlorolevulinate-modified bovine porphobilinogen synthase and the Pb-IIcomplexed enzyme. Acta Crystallogr D. 1996; 52:419-21.
136. Shimoni-Livny L, Carrell H L, Wagner T, Katz A K, Afshar C, Mitchell L W, Volin M, Jaffe E K, Glusker J P. Crystallization and preliminary X-ray diffraction studies of E-coli porphobilinogen synthase and its heavy-atom derivatives. Acta Crystallogr D. 1998; 54:438-40.
137. Kervinen J, Jaffe E K, Stauffer F, Neier R, Wlodawer A, Zdanov A. Mechanistic basis for suicide inactivation of porphobilinogen synthase by 4,7-dioxosebacic acid, an inhibitor that shows dramatic species selectivity. Biochemistry. 2001; 40(28):8227-36.
138. Jaffe E K, Kervinen J, Martins J, Stauffer F, Neier R, Wlodawer A, Zdanov A. Species-specific inhibition of porphobilinogen synthase by 4-oxosebacic acid. Journal of Biological Chemistry. 2002; 277(22):19792-9.
139. Ramirez U D, Minasov G, Focia P J, Stroud R M, Walter P, Kuhn P, Freymann D M. Structural basis for mobility in the 1.1 A crystal structure of the NG domain of Thermus aquaticus Ffh. J Mol Biol. 2002; 320(4):783-99.
140. Ramirez U D, Freymann D M. Analysis of protein hydration in ultrahigh-resolution structures of the SRP GTPase Ffh. Acta Crystallogr D Biol Crystallogr. 2006; 62 (Pt 12):1520-34.
141. Ramirez U D, Focia P J, Freymann D M. Nucleotide-binding flexibility in ultrahigh-resolution structures of the SRP GTPase Ffh. Acta Crystallogr D Biol Crystallogr. 2008; 64 (Pt 10):1043-53. PMCID: 2631121.
142. Lawrence S H, Selwood T, Jaffe E K. Environmental contaminants perturb fagile protein assemblies. in preparation. 2012.

What is claimed is:

1. A method of modulating a multimeric phenylalanine hydroxylase, the method comprising: applying a composition comprising a compound adapted to modulate formation of a multimeric phenylalanine hydroxylase to form an active form; associating the composition with the multimeric phenylalanine hydroxylase; promoting the multimeric protein to assemble into the active form, and thereby activating the multimeric phenylalanine hydroxylase.

2. A method of affecting a multimeric protein comprising an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that:
(i) one conformation of said units determines a first quaternary isoform but does not allow formation of another quaternary isoform;
(ii) a different conformation of said units determines one of a different quaternary isoform, but does not allow formation of the first quaternary isoform;
(iii) the different conformations of said units are in an equilibrium; and
(iv) the conformation of said different quaternary isoforms influences a function of said multimeric protein,
the method comprising:
applying to the multimeric protein a composition comprising a compound adapted to affect formation of an active form of the multimeric protein;
associating the composition with an active form of the multimeric protein;
promoting the multimeric protein to assemble into the active form, thereby affecting the multimeric protein to form the active form, wherein said multimeric protein is phenylalanine hydroxylase.

3. The method of claim 2, wherein the unit is a member selected from the group consisting of a monomer, a dimer, a trimer, a tetramer, a hexamer, and an octamer.

4. The method of claim 2, wherein said multimeric protein is phenylalanine hydroxylase comprising four phenylalanine hydroxylase monomers.

5. A method of identifying a compound that modulates formation of a multimeric protein by binding at a site other than an active site of the multimeric protein comprising an assembly having a plurality of units, wherein each of said units comprises a first complementary surface and a second complementary surface and wherein the first complementary surface of one unit is associated with the second complementary surface of another unit, provided that the assembly is at least one of different quaternary isoforms, the method comprising: a) providing at least one multimeric protein with the biochemical function; (b) identifying a compound that binds to the protein; and (c) testing for the ability of the compound to affect the biochemical function of the multimeric protein phenylalanine hydroxylase.

6. The method of claim 5, wherein said biochemical function of said multimeric protein correlates to a human disease or condition.

7. The method of claim 5, wherein the effect of the compound on the biochemical function is selected from the group consisting of inhibition, activation, enhancement, modulation, binding, and allosteric effect.

8. A method of identifying a compound adapted to modulate a multimeric protein by binding to a binding site of said multimeric protein, wherein the multimeric protein comprises an equilibrium of assembly states, each assembly having a plurality of units, wherein each of said units comprises a first complementary surface and a second complementary surface and wherein the first complementary surface of one unit is associated with the second complementary surface of another unit, provided that the assembly is at least one of different quaternary isoforms on condition that:
(i) one conformation of said units determines a first quaternary isoform but does not allow formation of another quaternary isoform;
(ii) a different conformation of said units determines one of a different quaternary isoform, but does not allow formation of the first quaternary isoform;
(iii) the different conformations of said units are in an equilibrium; and
(iv) the conformation of said different quaternary isoforms influences a function of said multimeric protein,
the method comprising;
providing a test compound;
providing the multimeric protein;
contacting the multimeric protein with the test compound; and
measuring the equilibrium of units of the multimeric protein,
wherein the compound adapted to affect the multimeric protein by binding to a binding site of the multimeric protein is identified when it affects the multimeric protein by binding to a binding site of the multimeric protein and thereby affects an equilibrium of units of the multimeric protein phenylalanine hydroxylase.

9. The method of claim 8, wherein the unit is a member selected from the group consisting of a monomer, a dimer, a trimer, a tetramer, a hexamer, and an octamer.

10. The method of claim 8, wherein the compound is adapted to affect a function of said multimeric protein.

11. The method of claim 8, wherein the compound is bound to a quaternary isoform having a greater activity.

12. The method of claim 8, wherein the compound activates the enzymatic activity of the multimeric protein.

13. The method of claim 8, wherein the compound is an activator which promotes formation of an active form of the multimeric protein.

14. A method of treating a disease or condition by administering a therapeutically effective amount of the compound identified by the method of claim 12.

15. The method of claim 12, wherein the disease or condition is deficiency of PAH activity.

16. The method of claim 12, wherein the disease or condition is hyperphenylalaninemia or, in more severe forms, phenylketonuria.

17. An antibody which selectively binds PAH isoforms 4mer* and 2mer*.

18. An antibody which selectively binds PAH isoforms 4mer and 2mer.

19. A method for predicting whether a hyperphenylalaninemia or phenylketonuria disease patient will respond effectively to treatment with an agent, comprising determining the ratio of PAH isoforms 4mer* and 2mer* to PAH isoforms 4mer and 2mer in a sample and comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* in the sample is greater than the ratio of PAH isoforms 4mer and 2mer in a control, this is an indication that the patient will respond effectively to treatment with the agent.

20. A method for predicting whether a hyperphenylalaninemia or phenylketonuria disease patient will respond effectively to treatment with a compound, comprising:
(a) obtaining a sample from a patient/individual;
(b) contacting said sample with a detectably-labeled first antibody which selectively binds PAH isoforms 4mer* and 2mer*;
(c) contacting said sample with a detectably-labeled second antibody which selectively binds PAH isoforms 4mer and 2mer;
(d) incubating the components of steps (b) for a period of time and under conditions sufficient to form an immune complex between said first antibody which selectively binds PAH isoforms 4mer* and 2mer*;
(e) incubating the components of steps (c) for a period of time and under conditions sufficient to form an immune complex between said second antibody which selectively binds PAH isoforms 4mer and 2mer;
(f) optionally separating unbound antibody from said sample;
(g) determining the detectably-labeled PAH isoforms 4mer* and 2mer*;
(h) determining the detectably-labeled PAH isoforms 4mer and 2mer;
(i) determining the ratio of PAH isoforms 4mer* and 2mer* to PAH isoforms 4mer and 2mer in the sample;
(j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control,
wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will respond effectively to treatment with the compound.

21. A method for predicting the sensitivity of a hyperphenylalaninemia or phenylketonuria disease patient to treatment with an compound, comprising determining the ratio of PAH isoforms 4mer* and 2mer* to PAH isoforms 4mer and 2mer in the sample, comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control, wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will be sensitive to treatment with the compound.

22. A method for predicting the sensitivity of a hyperphenylalaninemia or phenylketonuria disease patient to treatment with a compound, comprising:
(a) obtaining a sample from a patient/individual;
(b) contacting said sample with a detectably-labeled first antibody which selectively binds PAH isoforms 4mer* and 2mer*;
(c) contacting said sample with a detectably-labeled second antibody which selectively binds PAH isoforms 4mer and 2mer;
(d) incubating the components of steps (b) for a period of time and under conditions sufficient to form an immune complex between said first antibody which selectively binds PAH isoforms 4mer* and 2mer*;
(e) incubating the components of steps (c) for a period of time and under conditions sufficient to form an immune complex between said second antibody which selectively binds PAH isoforms 4mer and 2mer;
(f) optionally separating unbound antibody from said sample;
(g) determining the detectably-labeled PAH isoforms 4mer* and 2mer*;
(h) determining the detectably-labeled PAH isoforms 4mer and 2mer;
(i) determining the ratio of PAH isoforms 4mer* and 2mer* to PAH isoforms 4mer and 2mer in the sample;
(j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control,
wherein when the ratio of PAH isoforms 4mer* and 2mer* to PAH isoforms 4mer and 2mer in the sample is greater than the ratio of PAH isoforms 4mer* and 2mer* to PAH isoforms 4mer and 2mer in a control, this is an indication that the patient will be sensitive to treatment with the compound.

23. A method for the determination of the ratio of PAH isoforms 4mer* and 2mer* to PAH isoforms 4mer and 2mer in a sample, which comprises determining the ratio of PAH isoforms 4mer* and 2mer* to PAH isoforms 4mer and 2mer in the sample, comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control.

24. A method for the determination of the ratio of PAH isoforms 4mer* and 2mer* to PAH isoforms 4mer and 2mer in a sample, which comprises:
(a) obtaining a sample from a patient/individual;
(b) contacting said sample with a detectably-labeled first antibody which selectively binds PAH isoforms 4mer* and 2mer*;
(c) contacting said sample with a detectably-labeled second antibody which selectively binds PAH isoforms 4mer and 2mer;
(d) incubating the components of steps (b) for a period of time and under conditions sufficient to form an immune complex between said first antibody which selectively binds PAH isoforms 4mer* and 2mer*;
(e) incubating the components of steps (c) for a period of time and under conditions sufficient to form an immune complex between said second antibody which selectively binds PAH isoforms 4mer and 2mer;
(f) optionally separating unbound antibody from said sample;
(g) determining the detectably-labeled PAH isoforms 4mer* and 2mer*;
(h) determining the detectably-labeled PAH isoforms 4mer and 2mer;
(i) determining the ratio of PAH isoforms 4mer* and 2mer* to PAH isoforms 4mer and 2mer in the sample;
(j) comparing the ratio of PAH isoforms 4mer* and 2mer* to PAH isoforms 4mer and 2mer in the sample to the ratio of PAH isoforms 4mer* and 2mer* to PAH isoforms 4mer and 2mer in a control.
